SEQUENCE LISTING (70 sequences)

SEQ ID NO: 1 - DNA, 518 bp, Artificial Sequence
  Synthetic Sequence
gnnnnnngctctgcgagtcttgtcgtacccagtgatatccccaagacctatggaggagatacaggttctcctgaaatatccttcactaaaatttttaataatgtttctaaggagttgcctgggaagaccttagattttcattttgatatatctgagtttgtgtattgccaagtctgttgtgagcccttccacaagttttgtttagaggagaacgagcgccctctggaggaccagctggaaaattggtgttgtcgtcgttgcaaattctgtcacgtttgtggaaggcaacatcaggctacaaagcagctgctggagtgtaataagtgccgaaacagctatcaccctgagtgcctgggaccaaacnnnnnnnnnaaaaananctattttnnnnccctnangctgntggtggnctacccttgnnnccanangttnnntnnnnngnngntcnatnnnnnnnngncgnaggnnnnnncgnncatncctngaangntcangnntgnaangnnatcgnnnnnttn

SEQ ID NO: 2 - DNA, 457 bp, Artificial Sequence
  Synthetic Sequence
nannnnntagttctaagcaaaaaattccagcagatggagtccacaggatcagagtggactttaaggaggattgtgaagcanaaaatgtgtgggagatgggaggcttaggaatcttgacttctgttcctataacacccagggtggtttgctttctctgtgccngtagtgggcatgtagagacaccnattattggaaacnaatatggtgatcagnacagtgcggctggaagaaatgggaaaccaaaagttattgctgtcactagaagtacttcctcaacttcttctggttctaattctaatgccttggttcctgttagttggaaaaggccacngttatcacagcgaagaacaagagaaaagctaatgaatgtgctttctctctgtggtccagaatctggcctcccaaagaacccatcagttgtattttcttctaatgaggatttggaagtcgntgannn

SEQ ID NO: 3 - DNA, 437 bp, Artificial Sequence
  Synthetic Sequence
nnnnaaaannnnnnncaganggagtccacaggatcagagtggactttaagannnnnnnnnttggaaacaaatatggngannnnnacagngcggntggaagaaatgggaaaccaaaagttattgctgncnctagaagtacttcctcaacttcttctggntctaattctaatgccttggntcctgttagttggaaaaggccacagttatcacagcnaaaaacaagagaaaagctaatgaatgngctttctctctgtggtccagaatctggcctcccaaagaacccntnanntgtattttcttctaatgaggatttggaagtcggngagaagaacaagagaaaagctaatgaatgtgctttctctctgtggtccagaatctggcctcccaaagaacccatcagttgtattttcttctaatgaggatttggaagtcggtga

SEQ ID NO: 4 - DNA, 653 bp, Artificial Sequence
  Synthetic Sequence
nnnnnnnaaattccagcagatggagtccacaggatcagagtggactttaagacaccaattattggaaacaaatatggtgatcagcacagtgcggctggaagaaatgggaaaccaaaagttattgctgtcactagaagtacttcctcaacttcttctggttctaattctaatgccttggttcctgttagttggaaaaggccacagttatcacagcgaagaacaagagaaaagctaatgaatgtgctttctctctgtggtccagaatctggcctcccaaagaacccatcagtgagtaatcatgaagacctgtgtcaaggtctnnnnnatcatgttattccttgaagaaatgatctgtctccttgcttgccttttctaatttaccttacttacggtattaactcagaatgaaaaaaagatttcttatctcttgtgacacctggcagccatgctaactatgtgttcaactagttaaacatgaagaccaaatttaagctgtgaattcattgaagaatgaggcctgcggtggctttggggtactgatatctcntacgcagagtgacaaatctgatggatggactttttttttttttnnancgggnnncnggnntgtttttnnctntaannggnnnnngnnnngcggnnnannnnannnn

SEQ ID NO: 5 - DNA, 36 bp, Artificial Sequence
  Synthetic Sequence
gatcaccctgagtgcctgggaccaaactaccccacc

SEQ ID NO: 6 - DNA, 27 bp, Artificial Sequence
  Synthetic Sequence
taaaagagcatcatgtgtataactcac

SEQ ID NO: 7 - DNA, 27 bp, Artificial Sequence
  Synthetic Sequence
caggcactcagggtgatagctgtttcg

SEQ ID NO: 8 - DNA, 27 bp, Artificial Sequence
  Synthetic Sequence
ccagactttcttcttctttgtgggttt

SEQ ID NO: 9 - DNA, 30 bp, Artificial Sequence
  Synthetic Sequence
agctgtttcggcacttattacactccagca

SEQ ID NO: 10 - DNA, 20 bp, Artificial Sequence
  Synthetic Sequence
gctgaaaccatggaaggaaa

SEQ ID NO: 11 - DNA, 20 bp, Artificial Sequence
  Synthetic Sequence
aaaaattcgcatggaggaga

SEQ ID NO: 12 - DNA, 20 bp, Artificial Sequence
  Synthetic Sequence
tctacaagtgccaggggtct

SEQ ID NO: 13 - DNA, 20 bp, Artificial Sequence
  Synthetic Sequence
aaagccaagcttctccatca

SEQ ID NO: 14 - DNA, 20 bp, Artificial Sequence
  Synthetic Sequence
ctcccaaagtgctgggatta

SEQ ID NO: 15 - DNA, 20 bp, Artificial Sequence
  Synthetic Sequence
gctgcagtgagccattatca

SEQ ID NO: 16 - DNA, 20 bp, Artificial Sequence
  Synthetic Sequence
ctggaaggaagcccttaaca

SEQ ID NO: 17 - DNA, 20 bp, Artificial Sequence
  Synthetic Sequence
tttggtggggtagtttggtc

SEQ ID NO: 18 - DNA, 20 bp, Artificial Sequence
  Synthetic Sequence
gaatgcaggcactttgaaca

SEQ ID NO: 19 - DNA, 20 bp, Artificial Sequence
  Synthetic Sequence
tcaccgacttccaaatcctc

SEQ ID NO: 20 - DNA, 36 bp, Artificial Sequence
  Synthetic Sequence
gatcgaactattgccattggagagagtgctgaggat

SEQ ID NO: 21 - DNA, 25 bp, Artificial Sequence
  Synthetic Sequence
aaccacctccggtcaataagcagga

SEQ ID NO: 22 - DNA, 26 bp, Artificial Sequence
  Synthetic Sequence
aattccagcagatggagtccacagga

SEQ ID NO: 23 - DNA, 24 bp, Artificial Sequence
  Synthetic Sequence
caggagaatgcaggcactttgaac

SEQ ID NO: 24 - DNA, 25 bp, Artificial Sequence
  Synthetic Sequence
ccacaggatcagagtggactttaag

SEQ ID NO: 25 - DNA, 60 bp, Artificial Sequence
  Synthetic Sequence
ccactcttggtgccttggtgccattgaagtggtctttcggaagccaagggaggaactttt

SEQ ID NO: 26 - DNA, 60 bp, Artificial Sequence
  Synthetic Sequence
ccactcttggtgccttggtgccattgaagttgtacatagcaatctcacagggttcctaaa

SEQ ID NO: 27 - DNA, 60 bp, Artificial Sequence
  Synthetic Sequence
ataattgttctgatctaaattctttatagttgtacatagcaatctcacagggttcctaaa

SEQ ID NO: 28 - DNA, 60 bp, Artificial Sequence
  Synthetic Sequence
tttttcagggtataattgttctgatctaaattctttatagttgtacatagcaatctcaca

SEQ ID NO: 29 - DNA, 60 bp, Artificial Sequence
  Synthetic Sequence
tttttcagggtataattgttctgatctaaagaaggaagttattgatcaagtattgtaagg

SEQ ID NO: 30 - DNA, 60 bp, Artificial Sequence
  Sythetic Sequence
ctttcggaagccaagggaggaacttttcaagaaggaagttattgatcaagtattgtaagg

SEQ ID NO: 31 - protein, 531 aa, Artificial Sequence
  Synthetic Sequence
IKSAEQLTTFLKHVVSVFKQSSSEGIHLEHHLSEVALQTALSCSSRHYAGRSFQIFRALKQPLTATTLSDVLSRLVETVGDPGEDAQGFVIELLLTLESAIDTLAETMKHYDLLSALSQTSYHDPIMGNKYAANRKSTGQLNLSTSPINSSSYLGYXXXXXXXXXXXXXIGDRRGDRRRSNTLDIMDGRINHXXXXXXXXXXXXXXEKGMYDVQSTTEPTNLMATIFWIAXXXXXXXXXXXXXXXXXXXXXXXXXXXXDKSESREKIENVQSKLKWTNFPGLQQLFLKGFTSASTQEMTVHLLSKLISVSKHTLVDPSQLSGFPLNILCLLPHLIQHFDSPTQFCKETASRIAKVCAEEKCPTLVNLAHMMSLYSTHTYSRDCSNWINVVCRYLHDSFSDTTFNLVTYLAELLEKGXXXXXXXXXXXXXXXXXXXDLSAAPAKQFNLEIIKIIGKYVQSPYWKEALNILKLVVSRSASLVVPSDIPKTYGGDTGSPEISFTKIFNNVSKELPGKTLDFHFDISETPIIGNK

SEQ ID NO: 32 - protein, 541 aa, Artificial Sequence
  Synthetic Sequence
VKSAEQLSCFLKHMVKVFADSYPQARIAERWAQTALQLGLSCSSRHYAGRCLQIFRALNVPINSRMLSDILSRLVETVAEQGEDMQGYVTELLLTLEAAVDSLDSDFRPLDVMKDIFKSTPNLNNKDGGPNSILPGKKSPSGMTPQSSNYSIPSHGRSTSYSVSYCGRKANNSPCDKQVELRNRSSGIDLERSVVCKFGVGGGGAGSALSRSRSAQSLKMLGDTATQDDKMTILAQLFWLSVSLLESDYEHEFMLALRLLTRVLHRLPLDRPDARDKVEKLQQQLKWTAYPGVHALLLKGCTHSATYEPTIILLSQFAPLLTLPVCDPTQSCAFPMNVIALLPYMLLHYEDANEICIRSAENIAQVSTELGAKLENLGTVMTLYSRKTFCKESFQWTKCVVKYLHDTYAHMGLHMVAFLIEVLEKGPQQVQVPVLNVIHCMLHYVDLSAPQAQSINADLLRAIGKYLDTAYWKDSLKILKLIVTRSSSLQVVPPSDGSSAGLSFSFYGAYPDSDMFCKKELAGRTMEFTFDVSQTPLIGRR

SEQ ID NO: 33 - DNA, 70 bp, Artificial Sequence
  Synthetic Sequence
ttcagggtataattgttctgatctaaattctttatagttgtacatagcaatctcacagggttcctaaaat

SEQ ID NO: 34 - DNA, 70 bp, Artificial Sequence
  Synthetic Sequence
aagtcccatattaacaagactagatttaagaaatatcaacatgtatcgttagagtgtcccaaggatttta

SEQ ID NO: 35 - DNA, 91 bp, Artificial Sequence
  Synthetic Sequence
cctgagcccactcttggtgccttggtgccattgaagtggtctttcggaagccaagggaggaacttttcaagaaggaagttattgatcaagt

SEQ ID NO: 36 - DNA, 91 bp, Artificial Sequence
  Synthetic Sequence
ggactcgggtgagaaccacggaaccacggtaacttcaccagaaagccttcggttccctccttgaaaagttcttccttcaataactagttca

SEQ ID NO: 37 - DNA, 47 bp, Artificial Sequence
  Synthetic Sequence
ttctgatctaaattctttatagttgtacatagcaatctcacagggtt

SEQ ID NO: 38 - DNA, 47 bp, Artificial Sequence
  Synthetic Sequence
aagactagatttaagaaatatcaacatgtatcgttagagtgtcccaa

SEQ ID NO: 39 - DNA, 46 bp, Artificial Sequence
  Synthetic Sequence
gaagtggtctttcggaagcaagggaggaacttttcaagaaggaagt

SEQ ID NO: 40 - DNA, 46 bp, Artificial Sequence
  Synthetic Sequence
cttcaccagaaagccttcgttccctccttgaaaagttcttccttca

SEQ ID NO: 41 - DNA, 20 bp, Artificial Sequence
  Synthetic Sequence
ttctgatctaaattctttat

SEQ ID NO: 42 - DNA, 24 bp, Artificial Sequence
  Synthetic Sequence
aagactagatttaagaaatatcaa

SEQ ID NO: 43 - DNA, 24 bp, Artificial Sequence
  Synthetic Sequence
ggaggaacttttcaagaaggaagt

SEQ ID NO: 44 - DNA, 20 bp, Artificial Sequence
  Synthetic Sequence
cttgaaaagttcttccttca

SEQ ID NO: 45 - DNA, 12 bp, Artificial Sequence
  Synthetic Sequence
ttctgatctaaa

SEQ ID NO: 46 - DNA, 12 bp, Artificial Sequence
  Synthetic Sequence
aagactagattt

SEQ ID NO: 47 - DNA, 11 bp, Artificial Sequence
  Synthetic Sequence
aagaaggaagt

SEQ ID NO: 48 - DNA, 11 bp, Artificial Sequence
  Synthetic Sequence
ttcttccttca

SEQ ID NO: 49 - DNA, 21 bp, Artificial Sequence
  Synthetic Sequence
ttctgatctaaagaaggaagt

SEQ ID NO: 50 - DNA, 21 bp, Artificial Sequence
  Synthetic Sequence
aagactagatttcttccttca

SEQ ID NO: 51 - DNA, 22 bp, Artificial Sequence
  Synthetic Sequence
gaagtggtctttcggaagcaag

SEQ ID NO: 52 - DNA, 26 bp, Artificial Sequence
  Synthetic Sequence
cttcaccagaaagccttcgttccctc

SEQ ID NO: 53 - DNA, 27 bp, Artificial Sequence
  Synthetic Sequence
agttgtacatagcaatctcacagggtt

SEQ ID NO: 54 - DNA, 23 bp, Artificial Sequence
  Synthetic Sequence
catgtatcgttagagtgtcccaa

SEQ ID NO: 55 - DNA, 15 bp, Artificial Sequence
  Synthetic Sequence
ccttggtgccattga

SEQ ID NO: 56 - DNA, 18 bp, Artificial Sequence
  Synthetic Sequence
ggaaccacggtaacttca

SEQ ID NO: 57 - DNA, 27 bp, Artificial Sequence
  Synthetic Sequence
agttgtacatagcaatctcacagggtt

SEQ ID NO: 58 - DNA, 23 bp, Artificial Sequence
  Synthetic Sequence
catgtatcgttagagtgtcccaa

SEQ ID NO: 59 - DNA, 42 bp, Artificial Sequence
  Synthetic Sequence
ccttggtgccattgaagttgtacatagcaatctcacagggtt

SEQ ID NO: 60 - DNA, 42 bp, Artificial Sequence
  Synthetic Sequence
ggaaccacggtaacttcaacatgtatcgttagagtgtcccaa

SEQ ID NO: 61 - DNA, 80 bp, Artificial Sequence
  Synthetic Sequence
agattaaaactttttaaagcagcagttatttttggactcattgaaatgaaatactctgacattgtgatgtcacactaatt

SEQ ID NO: 62 - DNA, 80 bp, Artificial Sequence
  Synthetic Sequence
agattaaaactttttaaagcagcagttatttttggactcaaggatgactctaataatttcacaatgtagcttggttttgt

SEQ ID NO: 63 - DNA, 80 bp, Artificial Sequence
  Synthetic Sequence
tatttaaaagaaaatctccacctttaattctttatttcttatattgactctaataatttcacaatgtagcttggttttgt

SEQ ID NO: 64 - DNA, 80 bp, Artificial Sequence
  Synthetic Sequence
gattccagtggatcctgccagatgtaagggacacccttgattctctttggttatacctgacttcaagactctttccagga

SEQ ID NO: 65 - DNA, 80 bp, Artificial Sequence
  Synthetic Sequence
gattccagtggatcctgccagatgtaagggacacccttgacacagtgaaaccccatctctactaaaaatacaaaaaatta

SEQ ID NO: 66 - DNA, 80 bp, Artificial Sequence
  Synthetic Sequence
gcagatcacaaggtcaggagattgagaccatcctcgctaacacagtgaaaccccatctctactaaaaatacaaaaaatta

SEQ ID NO: 67 - protein, 288 aa, Artificial Sequence
  Synthetic Sequence
DPAPKKSSSEPPPRKPVEEKSEEGNVSAPGPESKQATTPASRKSSKQVSQPALVIPPQPPTTGPPRKEVPKTTPSEPKKKQPPPPESGPEQSKQKKVAPRPSIPVKQKPKEKEKPPPVNKQENAGTLNILSTLSNGNSSKQKIPADGVHRIRVDFKEDCEAENVWEMGGLGILTSVPITPRVVCFLCASSGHVEFVYCQVCCEPFHKFCLEENERPLEDQLENWCCRRCKFCHVCGGQHQATKQLLECNKCRNSYHPECLGPNYPTKPTKKKKVWICTKCVRCKSCGS

SEQ ID NO: 68 - DNA, 8342 bp, Artificial Sequence
  Synthetic Sequence
ggatcctgccccaaagaaaagcagtagtgagcctcctccacgaaagcccgtcgaggaaaagagtgaagaagggaatgtctcggcccctgggcctgaatccaaacaggccaccactccagcttccaggaagtcaagcaagcaggtctcccagccagcactggtcatcccgcctcagccacctactacaggaccgccaagaaaagaagttcccaaaaccactcctagtgagcccaagaaaaagcagcctccaccaccagaatcaggtgagtgaggagggcaagaaggaattgctgacccacaagtactaacaaaaaagcactgatgtctcaaacagcatttgaaagcaggaaatgtatgatttgaagtcttcagttcaagaaaatcagctctctttctaactattatgtttaataataaagaaacagaaacaaaaaaaacagttaaattggaggtattgttttaatttcctgttcgaagcctagagtttaaatagttttttttttttttttctaatggccctttcttcacaggtcagtcagtactaaagtagtcgttgccagcatctgactgcaatttattctgaattttttaggtccagagcagagcaaacagaaaaaagtggctccccgcccaagtatccctgtaaaacaaaaaccaaaagaaaaggtgaggagagatttgtttctctgccatttctcagggatgtattctattttgtagggaaaagccttatccttgacttctatgtagatggcagtggaatttcttaaaattaagaaacttcaagtttaggcttttagctgggcacggtggctcacgctggtaatcccaacacttagtgaggctgaggtgggaggattgcttgaggccagcagttcaagaccagcctgggcaacatagcaagaccctgtctttatttaaaccaaaaaaaaaaaaagaagaagaagaagttagccaggcatggtggcagttgcgtgtagtcccaggtactcaggaggctgagatagaaggattgtcttgagcccaggaattcaaggctgtagtgagctatgattgtaccactgcagtccagcctgggtgacaaagcaaaacactgtctccaaaaaaaatttaggcttggcaaggcgcagcggctcacgcctgtgatcccagcactttgggaagccgaagcaggcagatcacttgaggtcaggagttggagaccagcctggccaacatggtgaaaccctgtctctactgaaaatacaaaaattagccggttgtggtagtgggtgcttggtaatcctagctacttgggaggctgaggcagggggaattgcctgaaacctgcgaggcggaggctgcagtgagccgagattgcatcattgcactctagcctggacaacagagctagactccatcccaaaaaaaaaaaaaaaaagtagccgggcacggtggctcacgcctgtaatcccagcactttgggaggccgaggcgggcggatcatgagggcaggagatcgagaccatcctggctaacacggtgaaaccctgtctctactaaaaatacaaaaaattagcccggcgaggtggcgggcgcctgtagtcccagctactcaggagagtgagccaggagaatggcgtgaacccggggggcggagcctgcagtgagccgagatcgcgccactgcactccagcttgggtgacaccgagactccgtctcaaaaaaaaataaaaagtttaggctttagcctgtttcttttttggtttcttccttgttgcttttcccttctttgtggccccacatgttctagcctaggaatctgcttattctaaaggccatttggcgtaattattttttgaccccaacatcctttagcaattatttgtctgtaaaaatcacccttccctgtattcactatttttatttattatggataaagagatagtgtggtggctcacatctataatcccagcactttgggggcccaaggcgggaggatcacttgagggcaggagctggagaccagcctgggcagcacagtgacacacagttgctataaaaaatttaaaacccaactaggcatggtggcatgcacctgtagtcccagctactcttgagaagctgaggcaggaggatcacgagcccacaaggtctaggctgcagtgagctgtgactgtgccactgtattgcagcctaggcaacaaagcaagacccagtctcttttaaaaaaaaattcaaagattattgtttatgttggaaacatgttttttagatctattaataaaatttgtcatttgcattattatctgttgcaaatgtgaaggcaaatagggtgtgattttgttctatattcatcttttgtctccttaggaaaaaccacctccggtcaataagcaggagaatgcaggcactttgaacatcctcagcactctctccaatggcaatagttctaagcaaaaaattccagcagatggagtccacaggatcagagtggactttaaggtaaaggtgttcagtgatcataaagtatattgagtgtcaaagactttaaataaagaaaatgctactaccaaaggtgttgaaagaggaaatcagcaccaactgggggaatgaataagaactcccattagcaggtgggtttagcgctgggagagctttggacagtgttgttaggtcactgtttgtgaactgactgcagaacatacataatgaaacattcctatccatcctgaggagtatcagaggaagtaattccttcacatggaaagtatcaaaccatgatgattccttgagtcagcaaaactgtaagagaaattcaatcccagtgtattttcgcaatatcttcactatgaattgaacaactaggtgagccttttaatagtccgtgtctgagattaaaactttttaaagcagcagttatttttggactcattgaaatgaaatactctgacattgtgatgtcacactaattttatgcttttcatccttattttccatccaaagttgtgtaattgtaaaactttcctaagtgacctttctctctccacaggaggattgtgaagcagaaaatgtgtgggagatgggaggcttaggaatcttgacttctgttcctataacacccagggtggtttgctttctctgtgccagtagtgggcatgtagaggtaaggcatcctgcttctttgtaccccaggaagtacataaatgattgatctggggatgagattactatagtctgttttgttggtatttagcaggtactattccctgtttaaaccagctaaagaaatgttttgaagtattttagagattttaggaaggaatctgctattagagtagcaaagttattgagagtgaaaagatcaataatcccatctctcttaaattcagtctttattagagttctgatctttctgttagatgtctaaataagagaaaaaattatacagtggtctattaaaagggatgctattgatggttattttatattgtatatcaaagcctcttcatctataaggagctcttaccaattaataagaaaaaggaatgacatccagaaaaaaaaataggcaaaagacagaaatagataattcacaaaattagaaataaatacatgttgggtggcagggggaggtgaagggagggtgtctgttttttagccctctagtgaccaaaaactggaaattaaagcatgataaaaaaagaatcctgaataaatggggactttctgttggtggaaagaaatatagattagttacaatctttctttctgagggaattatttggaaatatatatatctatctttaaaataggtatatcctctaacatagcaattgcacttcaaacacttatggatataattagataaattggcaaatctgtagatataaagaagtgttcatttcaatattgctcataataataaaaaactggaaacaacccgaaagtccatctatagggagcatgggttaaaataagcatagggcatatagctgggcacggtggctcacgcctgtaatcccagcactttgggaggccaaggcaggcggatcacaaggtcaggagatccagaccatcctggctaacacagtgaaaccccgtctctattaaaaatacaaaaaaattagccgggtgtggtggcgggcgcctgtagtcccagctactcgagaggctgaggcaggagaacggcatgaacccgggaggtggagcttgcagtgagccgagatcgccccactgcactcccgcctgggcgacagagcaagactccgtctcaaaaaaaaataaaagtgtagggcatatataatgccaaatatgaagtcctaaagataatatatattaatattattaggttggtccaaaagtaattgcagtaataacatggaaagatgtccatgacatatcactgagtgaaaagagcaggttacaagataatatataaagcacaatcccatcttagtttggaaaagtgtttttaaagtatatatctagaaaacaatctggaaggattcacaccaaaatattaagagtgtggttggattatgggtgacctttatttgtttctctggtttttttttttttaatctttctgagttttttggagtatgtaccacctttacaatgaggaaggaaaaagtagcacaattttaaataggaagcagtagtttgtcatttataagggacatatcctacatcctttacagttcttaaattcctggcagatacctctttggcttattacttaccacataagatatgtattcaaaggtggtaaagaaaatccacgtcgggtgcagtggctcacgcctgtaatcccagtactttgggaggctgacgcaggaggaccgcttgagctcaggagttcaagaccagcctgagcaccatagtgagacctcatctctactaaaaaaaaaataaaataccaggcatggtagcatgtgcctgtagtcccagctactctagtcccagctacttgggaggctgaggtgagaggatcacttgagcccaggagatcgaggctgcagtgagccattatcacgccactgcactccagcctgggcaactaagcaagaccctgtctcaaaaaaattttaaaaaatttaaaaaataagaaaatccaagctaggttgaaatctgaatgttgagcagtcagtgagacacaaactagctaagaaagtcaaccctgcccacttgccatttgaagttattactagcaaaattacaaattattgcctactattcatttactaagcaaatattctcttagtccctattacgaacaacttattgttctaagtgcagaagttcagatatcattgagactgagaatattcagtctacaagtgccaggggtctactgtatcctcttttccgtcttaatacagtgctttgcacccatatatatgccacccacaggaataactttttttatagcaccagtccttcaacttctgggattaaacagattttttttcagggtataattgttctgatctaaattctttatagttgtacatagcaatctcacagggttcctaaaatataaaatagagaatagcatgctgcctgcactgcactcctaaagcatgaccagtgcttgataaactctcctccatgcgaattttttaaactttttatgttgacatgatttcagacttacaaaaaaactatgagttgtacagagaattctaagtacccctcacccaaattccctaagtgttaatatgtttctctgtgtgtatatattttacaaaataacaaataaaatacatatacacattttacctgtagatacacatgtatctaaaaatttgagaacaagttggagacataaaccattttacctctaaatattttagtgtatatttttaaaaatcaaggacgttctcgtatttaaccatggtataattaccaaatcaggaaattaacacactgggacattactattatctgatctataggccttatttaggtttgaccaattgtcccaataattcctttatggcaaaagaaaattctggattatcctagttagtatttttgaaaatcctatatcaatatgaaaataacttatttctaaaattagaaatggaggctgggcgtggtggctcacgcctataatcccagcactttgggaggccgaggcaggcagatcacaaggtcaggagattgagaccatcctcgctaacacagtgaaaccccatctctactaaaaatacaaaaaattagccaggtgtggtgggacgcgcctgtgatcccagctactcaggagactgaggctggagaatcgcttgaacccaacaggcggagggttgcagtgagtcgagatcgcaccactgcaccccagcctgggcgacagcgagactccgtctcaaaaaaataaataaataaaaattaaaacaattaaaaaaataaaattacaaatggaaaggacaaaccagaccttacaactgtttcgtatattacagaaaacgtttaaaccctccctatttcccccaccccactcctttatattcccatagctctttgtttataccactcttaggtcacttagcatgttctgttaaatcttgtattatatttattttgttactttctatttccactggtattaccactttagtactctgaatctcccgcaatgtccaatactgtacttttttacatagtcattgcttaatgaatatgtattgaattaaatatatgccagtggactactaaaacccaaagtatataagaagggtatggttgattatgtttttctacatattatttgacatacttctatcttcccatgttcttactatagtttgtgtattgccaagtctgttgtgagcccttccacaagttttgtttagaggagaacgagcgccctctggaggaccagctggaaaattggtgttgtcgtcgctgcaaattctgtcacgtttgtggagggcaacatcaggctacaaaggtacaaaacttggtaatagaactacagctgggcctctgtatcagtgggttctgtatccctggactcaaccaaccttggattgaatgtatctgggaaaaaatgagtagttgcctctgtactctatgtgaacagactttttcttgtcattatttcctaaacaatacagtataacaactatttacattgtattaggtatgataagtaatctagagataatttaaagtatatggtgggcggatcacttgaagccaggagttcgagaccagcctgagccaacatggtgaaaccccatctctactaaaaatacaaaaaattagccaggtgtggtggtgggcacctgtagtcccagctacttgggaggctgagggaggaaaatcgcttgaactttggaggcagaggttgcagtgagccactccagcctgtggtgcagtctgtcactccagcctgggtgacacagtgagactccatctcaaaaaaaaaaaaaaaaaaaaaactatatgggaggatgtgcattttgttatatgcaaatgctgcaccattttgtctagggacttgggcatccatggactttggtatcctctgggggtcctggaaccaatcccccatggaaaccaaggatgactgtgcttagagtattgctttctttcttgatttgtatttctgtcttccagttaagattttgtatctatattatttctctttttacttagtctgtctttagcatttaattgggtgtaatcagttgcctattttgtgttttaattttgggactatagcagaaaacatgatgttgaataaaattccaaaaataagtcaaatctacctaatatgaatactcatcactgagtgcctttggcaggaaataaatctatctcaatgcgttaattgggagtaaataatgcatgaggaaatttaaactcataattgtgtgctgtacttacttgccagtaaatgtgaaatggggtactaagtaataggtgttgggtgaaggtaatatgatgcttatctttttgccattatattttcttacagcagctgctggagtgtaataagtgccgaaacagctatcaccctgagtgcctgggaccaaactaccccaccaaacccacaaagaagaagaaagtctgggtgagttatacacatgatgctcttttatagagaaccaccatgtgactattggacttatgtaacttgtattacaaatatctatgcatgaggatgtcagtatgacaatctttttccctcattactaggaaatcatctcaggagagaaattaaatctataaatggatgcatttaagatctttttagttaagtaaagatattaaaaacaagaaattcctattgaatttcttttcttcttttctagatctgtaccaagtgtgttcgctgtaagagctgtggatcc

SEQ ID NO: 69 - protein, 1236 aa, Artificial Sequence
  Synthetic Sequence
IKSAEQLTTFLKHVVSVFKQSSSEGIHLEHHLSEVALQTALSCSSRHYAGRSFQIFRALKQPLTATTLSDVLSRLVETVGDPGEDAQGFVIELLLTLESAIDTLAETMKHYDLLSALSQTSYHDPIMGNKYAANRKSTGQLNLSTSPINSSSYLGYNSNARSNSLRLSLIGDRRGDRRRSNTLDIMDGRINHSSSLARTRSLSSLREKGMYDVQSTTEPTNLMATIFWIAASLLESDYEYEYLLALRLLNKLLIHLPLDKSESREKIENVQSKLKWTNFPGLQQLFLKGFTSASTQEMTVHLLSKLISVSKHTLVDPSQLSGFPLNILCLLPHLIQHFDSPTQFCKETASRIAKVCAEEKCPTLVNLAHMMSLYSTHTYSRDCSNWINVVCRYLHDSFSDTTFNLVTYLAELLEKGLSSMQQSLLQIIYSLLSHIDLSAAPAKQFNLEIIKIIGKYVQSPYWKEALNILKLVVSRSASLVVPSDIPKTYGGDTGSPEISFTKIFNNVSKELPGKTLDFHFDISETPIIGNKYGDQHSAAGRNGKPKVIAVTRSTSSTSSGSNSNALVPVSWKRPQLSQRRTREKLMNVLSLCGPESGLPKNPSVVFSSNEDLEVGDQQTSLISTTEDINQEEEVAVEDNSSEQQFGVFKDFDFLDVELEDAEGESMDNFNWGVRRRSLDSIDKGDTPSLQEYQCSSSTPSLNLTNQEDTDESSEEEAALTASQILSRTQMLNSDSATDETIPDHPDLLLQSEDSTGSITTEEVLQIRDETPTLEASLDNANSRLPEDTTSVLKEEHVTTFEDEGSYIIQEQQESLVCQGILDLEETEMPEPLAPESYPESVCEEDVTLALKELDERCEEEEADFSGLSSQDEEEQDGFPEVQTSPLPSPFLSAIIAAFQPVAYDDEEEAWRCHVNQMLSDTDGSSAVFTFHVFSRLFQTIQRKFGEITNEAVSFLGDSLQRIGTKFKSSLEVMMLCSECPTVFVDAETLMSCGLLETLKFGVLELQEHLDTYNVKREAAEQWLDDCKRTFGAKEDMYRINTDAQQMEILAELELCRRLYKLHFQLLLLFQAYCKLINQVNTIKNEAEVINMSEELAQLESILKEAESASENEEIDISKAAQTTIETAIHSLIETLKNKEFISAVAQVKAFRSLWPSDIFGSCEDDPVQTLLHIYFHHQTLGQTGSFAVIGSNLDMSEANYKLMELNLEIRESLRMVQSYQLLAQAKPMGNMVSTGF

SEQ ID NO: 70 - DNA, 5770 bp, Artificial Sequence
  Synthetic Sequence
ataaagagtgctgaacagttaactacatttttgaaacatgtggtttctgtttttaagcagtcaagctcagaaggaattcatctggaacatcatcttagtgaagttgctctgcaaacagcactttcctgttcttctcgacactatgctgggagatcctttcagattttcagggccctaaagcagcctctcactgcaactacactttctgatgttctctccagacttgtagaaactgtaggggatccaggagaagatgcacagggatttgtgattgagcttcttctcacattggaatctgcaattgatactttggctgaaaccatgaagcattatgatcttctttctgccctttctcaaacctcatatcatgatcctataatgggaaacaagtatgcagctaacaggaaaagcactggacaactcaatctaagcacaagtcccattaatagtagcagttatttgggatataacagtaatgcaagaagtaactctttgagattaagtttgattggtgaccgacgaggtgaccggcggcggagtaacacactggatataatggatggacggataaaccatagcagtagtttagcaaggactagaagcctttcctctctaagagagaaaggaatgtatgacgtgcagtccactactgagcctaccaacttgatggccaccattttttggatagcagcatctttattagaatcagattatgaatatgaatacctcctggctctcaggcttctcaacaaactgcttatccatttgcctttggataaatcagagagtcgagagaagattgaaaatgtacaaagcaaattgaaatggactaattttccaggacttcagcagctcttccttaagggttttacctcagcatctacacaagaaatgaccgtgcacctcctcagtaaactcatttctgtctccaaacatacattggtggatccttcccaattgtcaggctttcctcttaacatcctttgcttattgcctcacttaatccagcattttgacagcccaactcagttttgcaaagaaacagctagtcgaatagcaaaggtttgtgcagaagaaaaatgcccaacacttgtcaatctggcacacatgatgagtttgtacagtacacacacgtattccagagactgttctaactggatcaatgtcgtgtgcagatacctgcatgactccttctcagatacaacatttaatcttgtgacttatcttgcagagctgttagagaaaggattgtccagtatgcagcaatcattactacagattatttatagtctattgagtcatattgacctgtctgcagccccagccaagcagtttaatctggagatcataaagattattggcaaatatgtacagagtccttactggaaggaagcccttaacatattaaagctggtggtgtcacgctctgcgagtcttgtcgtacccagtgatatccccaagacctatggaggagatacaggttctcctgaaatatccttcactaaaatttttaataatgtttctaaggagttgcctgggaagaccttagattttcattttgatatatctgagacaccaattattggaaacaaatatggtgatcagcacagtgcggctggaagaaatgggaaaccaaaagttattgctgtcactagaagtacttcctcaacttcttctggttctaattctaatgccttggttcctgttagttggaaaaggccacagttatcacagcgaagaacaagagaaaagctaatgaatgtgctttctctctgtggtccagaatctggcctcccaaagaacccatcagttgtattttcttctaatgaggatttggaagtcggtgaccaacagactagcctaatttctacaacagaagacataaatcaagaggaagaagtagctgtggaagataatagcagtgaacaacagtttggtgtttttaaggattttgactttttagatgttgaattggaagatgcagagggtgaaagtatggacaatttcaactggggagttcgcaggcgctcactggacagtattgacaaaggggacactccatccctccaggagtaccagtgctctagtagcacccccagcctgaacctcaccaatcaggaggatacagatgagtcctcggaagaagaagcggcacttacagcaagccagatactctcacgcacacagatgttaaacagtgattctgccactgatgaaacaataccagaccatcctgacttacttctccagtctgaagattccactggcagcatcacaacagaggaagtgcttcaaatcagggatgagaccccaactttggaggcttctctagataatgctaacagccggctgcctgaggatacaacttcagtattaaaggaggaacatgttacaacctttgaagatgaaggatcctatataattcaagaacagcaggaatctcttgtgtgtcaaggaattcttgatttagaagaaactgaaatgccagagcctctagctcctgaaagttaccccgagtcagtctgtgaagaggatgttaccttagctctgaaagagctagatgaaagatgtgaagaagaagaagcggatttctccggactgtctagtcaagatgaagaagagcaagatggttttccagaagtacagacgtcgcctctgccgtcaccatttctttctgccatcatagccgcctttcagcccgtggcatatgatgatgaagaggaagcctggcgctgccacgtcaatcagatgctgtctgacaccgacgggtcctctgcagtgtttacttttcatgtgttttctaggctgtttcagacaattcaaagaaagtttggagaaataactaatgaggcagtcagctttcttggtgatagtctgcaacgcattggtaccaaatttaaaagttccttggaagtgatgatgctgtgttcagaatgcccaacagtctttgtggatgctgaaacactgatgtcatgtggtttgctggaaacactcaagtttggtgttttggagttgcaagaacacctggatacatacaatgtgaaaagagaagccgctgagcagtggctagatgattgtaagaggacatttggtgccaaagaagacatgtataggataaacacagatgcacaacaaatggaaatactagcagaattggagctctgccgaagattatacaaattgcattttcaattgctgcttctgttccaggcctactgtaaacttatcaaccaagtaaatacgataaaaaatgaagcagaggtcatcaacatgtcagaggaacttgcccaactggaaagtatcctcaaagaagctgagtccgcttccgaaaacgaagaaattgacatttccaaagctgcacaaactactatagaaactgccattcattctttaattgaaactttgaaaaataaagaatttatatcagctgtagcacaagtcaaagctttcagatctctctggcccagtgatatctttggcagttgtgaagatgaccctgtacagacactgttacatatatatttccatcatcagacgctgggccagacaggaagctttgcagttataggctctaacctggacatgtcagaagccaactacaaactgatggaacttaatctggaaataagagagtctctacgcatggtgcaatcataccaacttctagcacaggccaaaccaatgggaaatatggtgagcactggattctgagacacttcaggcctttaggaaagaaactaaactgaagatgatgaagaatattaaccaagcaccttttatggacccttgcattcactgataactttctggcagcatctactttttagtgtaactaatgtcaaactgtatcatcaaaaacaaagatctgaaagaaaaaaacatctgatattttaacagctgccaatatctcccacaataactgcgtgaagaaggaattttttaattacttaacctacgtgaaaagaaaagggctaaaagtgatgcctacaaatacattactttctggggaaagaaaagaattccaagaatgtttgcaataatggcctccaatactgaaacaaccaaaagcaggtgaaatgaggctgaaatcaaggctgtttcattttagctgaagacctgcaaagctgcttggatttctagcagttgaaaacctaacctgcaattgatgcttaatcatccatccacatgaacatgcagtcagctatacgcatcaagactcgtttgcaaaaaccattgatttttcagtatgtgggataagggtttgggtttttttgttttttggtctgtataaggaattatgtgtgtgtgagttgggatgtatggatatgtgatagtcatattttcaaggtatggatgtttggtgataacgtctcagagcatgcctaaaagagcactgcaagattatttttgaagaaatgttattttattagatctaactcttcatagtatgtaaatgttagaacattttaataatattttaaaactgggctttcccagtatttcaaaaagaaataatatatctgttaacgttattggaatgctgctcagttctctgatcagtgcttatgttatgattgttgataactaaccaaagtagatgcctgcagagactttaaaatgtaaaataaagatgtatgctgcctgtcagctattctcattagaaaagttttaacttattttactccatatagaaactgtagagactaaaaccagttattttcttgtacatttgtgccatgcactgttgttatacaagaataggtgtacaaagctaaagaaaaattgtggtcattgatgatggaatatatgacttgcaggctttgaagtctgcagaattcaagaaaagagctgcaaatgcatttttgtatctttattcaggactcacatgctttaccctaaagaaacctggggctaggggaaatgaaaggaagcctgaagactgactaccaaaacatgcaatatacttattcactgtctaagtctgtagtataacatgaactggagtctctatccttttctaaaatcgcattttgtaagaaaagaaagaaaaaaaaaaaaaaagaatgtagtcccacaactcctgatttgaaagaaacaccttgtattttttatatacatctcttatccactgtgacttttcttttaaactgggctctgtgattccagagtttagaatgtagaaatgaaatgcttagctttaccttttcttggggggtgtggaccctgccagaaatgtaattacgtatgctcaagtgcattaattgaaggcactgtgcacaccattggactgctgaccacagttaaacatttccagtaaaatttcgtaactggtcacagctcattcataatcactgtattttttgaccctgataaagacaattattttgatggtgttctggtactgtaattggtatcctttaaattatttaaaacttttgggtgtggggcaggcagagagggatggtgtccagagatacatcacactttacatcctatcttactgtcctctgtttttcttaccctccttacatcctttttgttaaaaataaaaagcattgtagctgttggtttgtgttgtattttaacaaagaatgaatgattacagtgtgcatcatttagaaacctttcaaatgttaccttcttgaattgccctagaaaagaacaaaattagttgactacccaaccaaacctttcagttgatatttgctttttgtgtttatttatatgtaactaagtctattaatataataaaaatgaatatacctgcagc

(19) United States
(12) Patent Application Publication
(10) Pub. No.: US 2006/0063179 A1
(43) Pub. Date: Mar. 23, 2006

(54) MIFL- a new partner gene of MLL and methods of use thereof

(76) Inventors: Carolyn A. Felix, Ardmore, PA (US); Blaine Walker Robinson, East Lans downe, PA (US); Nai-Kong Cheung, Purchase, NY (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/199,544

(22) Filed: Aug. 8, 2005

Related U.S. Application Data
(60) Provisional application No. 60/599,385, filed on Aug. 6, 2004.

(51) Int. Cl. C12Q 1/68; C07H 21/02; C12P 21/06; C07K 14/82

(52) U.S. Cl. 435/6; 536/23.1; 435/69.1; 435/320.1; 435/325; 530/350

(57) ABSTRACT

Compositions and methods are provided for the identification of partner genes with MLL. Also provided is a new partner gene of MLL termed MIFL.

This application claims the benefit of U.S. Provisional Application No. 60/599,385, filed Aug. 6, 2004, the entire disclosure of which is incorporated by reference herein.

Pursuant to 35 U.S.C. §202(c) it is acknowledged that the U.S. Government has certain rights in the invention described herein, which was made in part with funds from the National Institutes of Health, Grant Numbers RO1 CA77683, CA85469, and CA80175.

### FIELD OF THE INVENTION

This invention relates to the fields of molecular biology and oncology. More specifically, the present invention provides a new fusion gene, methods of use thereof and improved methods for isolating and characterizing such genes and their encoded proteins.

### BACKGROUND OF THE INVENTION

Several publications and patent documents are cited throughout the specification in order to describe the state of the art to which this invention pertains. Each of these citations is incorporated herein by reference as though set forth in full.

The estimated frequency of developing a secondary cancer after surviving a primary cancer is about 7%. Leukemia is the major secondary cancer arising from chemotherapy involving alkylating agents and DNA topoisomerase II inhibitors. In cases where DNA topoisomerase II inhibitors, such as epipodophyllotoxins, anthracyclines, etoposide, and doxorubicin, have been used, MLL (myeloid/lymphoid or mixed lineage leukemia) translocations are a common occurrence at chromosome band 11q23 involving over 40 partner genes. The MLL gene at chromosome band 11q23 encodes a multi-domain oncoprotein that functions in a large macromolecular protein complex to regulate transcription in hematopoietic cells and other cell types (Nakamura et al. (2002) Mol. Cell, 10:1119-1128; Yokoyama et al. (2002) Blood, 100:3710-8; Hsieh et al. (2003) Mol. Cell. Biol., 23:186-94). Many of the more than 40 partner genes have structural motifs of nuclear transcription factors or transcriptional regulatory proteins. Other MLL partner proteins are cytoplasmic or cell membrane proteins.

Nearly all known MLL translocations have been associated with a leukemia phenotype in humans and, where studied, MLL fusion proteins from the der(11) chromosome cause leukemia in mice. Acute myeloid leukemia (AML) is the most common disease associated with treatment related MLL translocations and has a short latency period. The DNA topoisomerase II inhibitors/poisons most likely play a role in treatment-related leukemia but the mechanism is currently under investigation. The results of murine experiments have converged upon a model where the diverse MLL fusion proteins result in altered Hox gene regulation, which affects the incidence or phenotype of leukemia in mice (Yokoyama et al. (2004) Mol. Cell. Biol., 24:5639-49; Kumar et al. (2004) Blood, 103:1823-1828). It has further been suggested that the cooperation of mutations in each of two different complementation groups that confer either a proliferative and/or survival advantage to hematopoietic progenitors or impair their differentiation are required to cooperate for leukemogenesis (Gilliland et al. (2004) Hematology, 80-97). Translocations disrupting MLL impact the HOX family of transcription factors and fall into the complementation group affecting differentiation (Gilliland et al. (2004) Hematology, 80-97). MLL translocations often are accompanied by mutations of the FLT3 tyrosine kinase or overexpression of wild-type FLT3, which affects proliferation (Armstrong et al. (2004) Blood, 103:3544-6; Gilliland et al. (2004) Hematology, 80-97).

The ability to rapidly and accurately determine MLL translocations is crucial in cases involving infant leukemia and leukemias arising from DNA topoisomerase II inhibitor therapy. Determining the specific partner gene and identifying the sequence and locations of the genomic breakpoint junction in MLL may impact how the leukemia is treated or how and when it developed in the first place. There are well over 40 partner genes associated with MLL with more being cloned every year. The need to expand traditional PCR techniques used to identify these translocations and partner genes is highly desirable, since the leukemias of many patients have rearrangements that cannot be cloned with standard PCR-cloning strategies.

### SUMMARY OF THE INVENTION

The instant invention relates to a new genomic sequence discovered using a modified panhandle PCR approach. The new genomic sequence is a newly identified translocation between MLL and a new partner gene on chromosome 4 designated MIFL (MLL Insufficient for Leukemia). The translocation was identified by utilizing a panhandle PCR technique to clone the der(11) and der(4) genomic breakpoint junctions using BglII digested DNA from a patient who had neuroblastoma and developed a (4;11) translocation in the marrow during intensive chemotherapy.

In accordance with the instant invention, nucleic acid molecules are provided comprising a translocation joining MLL and MIFL (also referred to as KIAA0826 in GenBank). The fusion transcripts and protein products expressed from these nucleic acids also comprise aspects of the invention.

In one embodiment of the invention, a method for screening compounds for the ability to modulate proliferation and/or transformation of a cell is provided. An exemplary method entails providing cells comprising the MLL-MIFL nucleic acid of the invention; incubating the cells with at least one compound; and monitoring proliferation of the cells, wherein a change in the proliferation/transformation of the cells incubated with the compound(s) as compared to cells not treated with the compound is indicative of the ability of the compound to modulate cellular proliferation.

The invention also provides a method of immortalizing cells in culture via the introduction of the MLL-MIFL nucleic acid molecule of the invention. Preferably the MLL-MIFL nucleic acid is cloned within an expression vector. Cell lines of such cells are also within the scope of the invention.

In yet another aspect, a transgenic animal comprising the MLL-MIFL nucleic acid molecule is disclosed. Such transgenic mice may be used in methods for screening compounds for the ability to modulate leukemogenesis. An exemplary method entails providing a transgenic animal which comprises MLL-MIFL; administering the compound to the animal; and monitoring the animal for leukemogenesis.

Isolated primary host cells comprising the MLL-MIFL nucleic acid of the invention are also provided. Such cells are preferably obtained from a patient and may optionally be subjected to mutagenesis.

In yet another aspect of the invention, a method for the screening cells for genetic changes associated with the onset of leukemia is disclosed. An exemplary method entails passaging the cells comprising the MLL-MIFL nucleic acid in an immunocompromised host (e.g., a NOD-SCID mouse); assessing the mice for the development of leukemia; isolating said cells from mice which develop leukemia; and performing proteomic or genomic analysis comparing the passaged cells with cells isolated from said patient to identify proteomic or genetic changes associated with the onset of leukemia.

One other aspect of the invention entails a method for screening for MLL-MIFL fusion nucleic acid sequences in human subjects. Such a method can comprise isolating nucleic acid from cells obtained from said patient; contacting the nucleic acid with a probe which hybridizes to a MLL-MIFL nucleic acid sequence under conditions suitable for hybridization to occur and detecting hybridization if present, wherein hybridization indicates the presence of a MLL-MIFL fusion transcript in said patient. In an alternative approach, the screening method entails subjecting the isolated nucleic acid to BgII panhandle PCR.

### BRIEF DESCRIPTION OF THE DRAWINGS

FIG. 1 is a schematic of the treatment and clinical events in a patient diagnosed with neuroblastoma. FISH karyotypes, Southern Blot analysis, confirmation of MLL translocation, and the percentage of translocated MLL are identified throughout the timeline. VP16 (horizontal striped rectangles) denotes etoposide; CAV, cyclophosphamide/doxorubicin/vincristine; PVP, cisplatin/etoposide; 3F8, anti-G_{D2} antibodies (* represents radioactive); PBSCH, peripheral blood stem cell harvest; PBSCI, peripheral blood stem cell infusion; TCT, thiotepa/carboplatin/topotecan; GMCSF, granulocyte macrophage colony stimulating factor plus 3F8* (diamond inset rectangles); accutane (black rectangles). Scale indicates months (mos) from neuroblastoma diagnosis.

FIG. 2 is an image of a Southern blot analysis identifying MLL bcr rearrangements in patient NB142. BamHI-digested (lanes 1 and 2), BamHI/BglII-digested (lanes 3 and 4), and BglII-digested (lanes 5 and 6) DNA are detected with B859 fragment of ALL-1 cDNA. The dashes represent unrearranged MLL allele (e.g., 8.3 kb (lanes 1 and 2), 6.0 and 2.3 kb (lanes 3 and 4), and 6.0 and 2.9 kb (lanes 5 and 6)) and the arrows show rearrangements (3.2 kb fragment was from MLL-MIFL). A patient without MLL rearrangements is shown in lanes 1, 3, and 5. Lanes 2, 4, and 6 are from patient NB142.

FIG. 3A provides images of gels showing that the BglII panhandle PCR product (middle panel) was consistent with the 6.0 kb germline unrearranged MLL allele. BglII reverse panhandle PCR product (right panel) was consistent with the 3.2 kb rearrangement on the Southern blot (lane 6 of FIG. 2). FIG. 3B is a schematic of genomic breakpoint junction sequence of the other derivative chromosome in TOPO TA-PCR generated subclone from BglII reverse panhandle PCR. Comparison with normal KIAA0826 (GenBank accession no. AB020633) and MLL (GenBank accession number U04737) genomic sequences identified KIAA0826 and MLL breakpoint positions indicated by a dash and bold letters, which could not be assigned precisely because of identical AGT sequence in both genes. Short sequence homologies in KIAA0826 and MLL are underlined. FIG. 3C provides an image of a gel depicting that the clonotypic PCR produced the predicted size products of NB142 der(11) and der(4). Sequencing of the products confirmed the der(4) genomic breakpoint and identified the der(11) genomic breakpoint. FIG. 3D is a schematic of genomic breakpoint junction sequence of der(11) chromosome from a genecleaned clonotypic-PCR generated product. Comparison with normal KIAA0826 (GenBank accession no. AB020633) and MLL (GenBank accession number U04737) genomic sequences identified KIAA0826 and MLL breakpoint positions indicated by a dash and bold letters, which could not be assigned precisely because of identical AA sequence in both genes. Short sequence homologies in KIAA0826 and MLL are underlined.

FIG. 4A depicts sequences in TOPO TA PCR-generated subclones which were identified via cDNA panhandle PCR analysis of total RNA from patient NB142. Twenty-eight subclones contained MLL sequence only. Alternatively spliced transcripts were detected. FIG. 4B provides an image of a gel depicting RT-PCR analysis of total RNA from patient NB142 which revealed MLL-KIAA0826 and KIAA0826-MLL chimeric transcripts. RT-PCR reactions with sense and antisense primers from MLL exon 6 and KIAA0826 exon 11, respectively, and randomly primed cDNA template gave four products (lane 2). RT-PCR reactions with sense and antisense primers from KIAA0826 exons 7 and MLL exon 10, respectively, and randomly primed cDNA template gave a single 363 bp product (lane 4). Reactions using RNA-negative reagent control (dH₂O) are shown (lanes 3 and 5). FIG. 4C is a schematic drawing of direct sequencing which revealed in-frame fusion transcripts: (top) MLL exon 7 at position 4218 or MLL exon 8 at position 4332 of the MLL cDNA (GenBank accession no. L04284) to KIAA0826 exon 9 at position 1573 of the KIAA0826 cDNA (GenBank accession no. AB020633) and (bottom) KIAA0826 exon 8 at position 1572 of the KIAA0826 cDNA to MLL exon 9 at position 4333 of the MLL cDNA.

FIG. 5 provides images of gels depicting the PCR amplification of the der(11) and der(4) genomic breakpoint junctions with clonotypic primers in DNAs prepared from bone barrow aspirates on glass slides during neuroblastoma treatment. The der(11) and der(4) translocations were detected at 16 mos from neuroblastoma diagnosis and all specimens thereafter. Sequencing was performed on all samples to verify the translocation.

FIGS. 6A and 6B are gels of DNA obtained from a sample 21.5 months after neuroblastoma diagnosis with nearly 100% of the cells containing the MLL translocation which was used to determine the sensitivity of PCR using der(11) (FIG. 6A) or der(4) (FIG. 6B) clonotypic primers. The der(11) PCR product was detectable at 1/10000 dilution (10 pg) and the der(4) PCR product was detectable at 1/100 (1 ng). Control DNA from a normal patient and a reagent control (dH₂O) are shown (lanes 8 and 9, respectively).

FIG. 7 is an alignment of MIFL protein sequence and Drosophila fry protein depicting identical and homologous regions.

FIGS. 8A-8D provide images of autoradiographs depicting MLL bcr positions 5382-5570 (FIGS. 8A and 8B) or positions 1387 to 1424 in MIFL intron 8 (FIGS. 8C and 8D) identified utilizing topoisomerase II in vitro cleavage assays. Autoradiographs show cleavage products after 10 minute incubation at 37° C. of 25 ng (30,000 cpm) singly 5′ end-labeled DNA with 147 nM human DNA topoisomerase II, 1 mM ATP and, where indicated, 20 μM etoposide, etoposide catechol, etoposide quinone or doxorubicin (FIGS. 8A and 8C), or lower doxorubicin concentrations (FIGS. 8B and 8D). Specified reactions were incubated for an additional 10 minutes at 75° C. before trapping the covalent complexes (FIGS. 8A and 8C). The 5′ side (−1 position) of the cleavage sites are shown by the dashes at far right.

FIG. 9 provides a schematic model for repair of cleavage sites to form der(11) and der(4) genomic breakpoint junctions. Cleavage sites enhanced by etoposide or its metabolites were used to build models for creation of the breakpoint junctons. Topoisomerase II cleavage sites at MLL intron 8 and MIFL with 4-base 5′ overhangs are shown at top. The processing includes exonucleolytic digestion to form single-base homologies and create both breakpoint junctions of the t(4;11) by error-prone NHEJ (boxes). In formation of the der(11), certain nucleotides are lost by exonucleolytic digestion (middle) before NHEJ (middle) joins the indicated bases. Also, certain nucleotides of MLL are lost by exonucleolytic digestion (bottom) and the der(4) also forms by NHEJ (bottom).

FIG. 10 is a graphical representation of real-time PCR analysis of gene expression in NB142 marrow from 19 months after NB diagnosis, in which 82% of cells contained the translocation, compared to MLL(+) leukemia cases. Log10 (Relative Quantity) is equivalent to 2(−ΔΔC_{T}) where the endogenous control is 18s rRNA and the calibration sample (set to 1 for all genes) is NB142. Error bars represent standard deviation from triplicate experiments. The two t-AML cases had increased expression of both HOXA9 and MEIS1 co-factor compared to NB142, and expression of other leukemia associated genes (HOXA7, PBX3, FLT3, BCL2) were reduced in NB142 compared to the leukemia cases. Asterisks (*) represent any transcript that was undetectable.

FIG. 11A is a schematic drawing of the steps in BglII Panhandle PCR to identify the genomic breakpoint junction of the der(11) chromosome of MLL translocation. FIG. 11B is a schematic drawing of the steps in BglII Reverse Panhandle PCR to identify the genomic breakpoint junction of the other derivative chromosomes of MLL translocation. Corkscrew arrow indicates the genomic breakpoint junction between MLL and the partner gene.

FIG. 12 provides images of gels showing the MLL bcr rearrangements in AML of patient 21 (BamHI-digested DNA, left; BglII-digested DNA, right). The 8.3 kb fragment (left) and 6.0 kb and 2.9 kb fragments (right) on Southern blot are from unrearranged MLL alleles; arrows show rearrangements. Both blots were probed with a cDNA derived probe, B859, involving exons 5-11 between the BamHI sites in the MLL bcr.

FIG. 13 provides an image of a gel depicting the BglII panhandle PCR product from der(11) chromosome of t(6;11)(q27;q23) in AML of patient 21 (left). Also provided is a schematic of the der(11) genomic breakpoint junction in TOPO TA cloning PCR-generated subclones from BglII panhandle PCR (right). One subclone (5114 bp) was sequenced in its entirety to determine the genomic breakpoint junction and partner gene. Short homologies are underlined. Dashes represent the base pair where the der(11) breakpoint junction occurs. The ‘AGGA’ bp insertion is between a precise genomic breakpoint junction.

FIG. 14 is a schematic drawing of the MLL bcr. BamHI and BglII restriction enzyme cut sites are shown throughout the bcr (exons 5-11). The B859 probe encompasses the bcr, exons 5-11, and is used for Southern blot.

FIG. 15 provides an image of a gel depicting BglII panhandle PCR product from der(4) chromosome of t(4;11)(q21;23) in ALL of patient 45 (left). Also provided is a schematic of the der(4) genomic breakpoint junction in TOPO TA cloning PCR-generated subclones from BglII panhandle PCR (right). One subclone (3470 bp) was sequenced in its entirety to determine the genomic breakpoint junction and partner gene. Short homologies are underlined. Dashes represent the base pair where the der(4) breakpoint junction occurs. The “A” residue in both genes precluded a precise translocation.

FIGS. 16A-16D provide the MLL-MIFL and MIFL-MLL transcripts (SEQ ID NOs: 1-4, respectively) depicted in FIG. 4C.

FIGS. 17A-17C provide the sequence of MLL, Gene Bank Accession Number U04737.

FIGS. 18A-18C provide the sequence of MIFL, Gene Bank Accession number AB020633.

### DETAILED DESCRIPTION OF THE INVENTION

Epipodophyllotoxins, anthracyclines and other chemotherapeutic DNA topoisomerase II poisons are associated with MLL-rearranged leukemias and, less often, leukemias with different balanced translocations. Every MLL translocation identified so far in patients has been associated with leukemia and, where tested, murine models have established that the der(11) protein products are leukemogenic. Described herein is a highly novel t(4;11)(p12;q23) translocation that emerged in the bone marrow that confers a proliferative advantage but not a leukemia phenotype in a patient with stage 4 neuroblastoma (NB) following N8 therapy, which consisted of 3 cycles of cyclophosphamide/doxorubicin/vincristine, 2 cycles of cisplatin (P)/etoposide (VP), surgery, local radiation, anti-G_{D2} monoclonal antibody (3F8), myeloablative therapy (thiotepa, carboplatin, topotecan) plus autologous PBSC transplant (harvested after 1 cycle of PVP), followed by 7 more cycles of 3F8 plus GM-CSF, 4 cycles of oral VP, and 5 cycles of accutane. Cumulative doses of doxorubicin and VP were 225 mg/m² and 5.4 g/m² (4.2 g/m² orally), respectively. Seventeen months after NB diagnosis (11.5 months after transplant) at completion of oral VP, surveillance FISH showed t(4;11)(p12;q23) in 10.5% of marrow cells. The patient is now 32 months from NB diagnosis and 1 year off all therapy. Although both karyotype and FISH showed t(4;11) in 82-100% cells in 7 serial subsequent marrows, all had normal morphology and there is no clinical evidence of leukemia. Molecular analyses of sequential bone marrow samples were undertaken to characterize the translocation. Southern blot analysis of the MLL bcr in BamHI and BglII digested DNAs showed two rearrangements consistent with both derivative chromosomes. A new BglII-based reverse panhandle PCR identified the der(4) breakpoint junction sequence in a 3.2 kb BglII genomic rearrangement and provided the sequence to isolate the der(11) genomic breakpoint junction by clonotypic PCR. The partner DNA was a novel gene localized to chromosome band 4p12. The translocation fused MLL intron 8 with intron 8 in the new partner gene, and occurred with a loss of 8-13 bases from MLL and 31-36 bases from the partner gene. Both breakpoint junctions contained short homologous sequences suggesting NHEJ. By clonotypic nested PCR (sensitivity 1 cell in 10⁴-10⁵ cells) the translocation was absent at NB diagnosis, and was first detectable at the completion of oral VP (16 months after neuroblastoma diagnosis). RT-PCR revealed in-frame der(11) and der(4) fusion transcripts. Real-time PCR analysis of known MLL target genes and differentially expressed genes in MLL-rearranged leukemias revealed low levels of HOXA4,A5,A7,A9, FLT3, MEIS1, and PBX3 compared to other treatment-related leukemias. Temporal emergence of this translocation after the high total VP dose is of interest because others recently found that cumulative doses of VP>6 g/m² carry an especially high risk of leukemia, raising concern about the role of the oral VP in the genesis of the translocation. This study indicates that the different partner genes of MLL do not confer the same leukemia potential. Because the t(4;11)(p12;q23) confers a proliferative advantage but is insufficient for leukemia, the new partner gene has been designated MIFL (MLL Insufficient for Leukemia), however it remains to be determined whether any relevant secondary alteration will result in leukemic transformation.

The most common molecular abnormalities in acute leukemias of infants and acute leukemias related to chemotherapy with DNA topoisomerase II inhibitors are translocations involving the breakpoint cluster region of the MLL gene at chromosome band 11q23. Panhandle and reverse panhandle PCR techniques use primers derived from MLL that are able to amplify the genomic breakpoint and determine the unknown partner gene by adding a MLL oligonucleotide sequence to the unknown partner gene and forming a panhandle structure prior to the PCR amplification. These techniques enable the determination of an unlimited number of potential partner genes and various genomic breakpoint junctions in the breakpoint cluster region of MLL. Cloning of MLL genomic breakpoints has been successful using BamHI-digested DNA, but many translocation breakpoint junction sequences occur on restriction fragments that may be too large to clone using this technique under conditions and with reagents that are used routinely. BglII-digested DNA shortens the breakpoint cluster region of MLL by 2.3 kb and may decrease the section of partner gene associated with MLL thus enabling the amplification of genomic breakpoints and the potential of discovering novel partner genes on restriction fragments previously thought too large using BamHI-digested DNA. Two patients that have had their genomic breakpoints and partner genes identified using BamHI-digested DNA have been used as test cases to determine the validity and feasibility of BglII panhandle and reverse panhandle PCR. Patient 21 demonstrated a 5.2 kb rearrangement on Southern blot and was cloned using BglII panhandle PCR. This technique identified the partner gene, AF-6, with a MLL genomic breakpoint of position 2913 in intron 7. Patient 45 demonstrated a 3.7 kb rearrangement on Southern blot and was cloned using BglII reverse panhandle PCR. This technique identified the partner gene, AF-4, with a MLL genomic breakpoint of position 6166 or 6167 in intron 8. Both patients' genomic breakpoints were identical to ones previously cloned using BamHI-digested DNA. BglII panhandle and reverse panhandle PCR provide a whole new dimension to the previously unclonable breakpoints and partner genes for which the rearrangements may be too large for BamHI.

In accordance with one embodiment of the invention, transgenic animals comprising a nucleic acid encoding for the MLL-MIFL translocation are provided. The term “transgenic animal” is intended to include any non-human animal, preferably vertebrate, in which one or more of the cells of the animal contain heterologous nucleic acid encoding the MLL-MIFL translocation. Non-human animals include without limitation, rodents, non-human primates, sheep, dog, cow, amphibians, zebrafish, reptiles, and the like. In a preferred embodiment, the animal is a mouse.

The heterologous nucleic acid is introduced into the animal by way of human intervention, such as by transgenic techniques well known in the art (see, e.g., Ausubel et al. (Ed.), Current Protocols in Molecular Biology (2005) John Wiley & Sons, New York). For example, the transgenic non-human animals of the invention are produced by introducing the nucleic acid into the germline of the non-human animal. Embryonic stem cells (ES) are the primary type of target cell for introduction of the nucleic acid molecule into the non-human animal in order to achieve homologous recombination. ES cells may be obtained from pre-implantation embryos cultured in vitro and fused with embryos (see, e.g., Evans et al. (1981) Nature 292:154-156; Bradley et al. (1984) Nature 309:255-258; Gossler et al. (1986) Proc. Natl. Acad. Sci., 83:9065-9069; Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240, 1468-1474. The transfected embryonal cells may be incubated in vitro for varying amounts of time, or reimplanted into the surrogate host, or both. Transgenic offspring of the surrogate host may be screened for the presence and/or expression of the transgene by any suitable method (e.g., Southern blot, Northern blot, or Western blot analysis).

Alternatively, murine bone marrow cells can be used for methylcellulose culture assays of in vitro immortalization of primary hematopoietic cells by MLL-MIFL. Constructs to be tested will include full length MLL-MIFL fusion constructs. Control constructs will include 1) vector without insert, 2) 5′-MLL to exon 7 lacking in the partner gene, 3) MIFL, and 4) full length MLL-ELL with known immortalizing properties as a positive control. Each cDNA will be cloned into an internal ribosomal entry site (IRES) G418 retroviral vector. The retroviral vectors will be used to transfect Bosc23-derived packaging cells to produce helper free (unable to replicate) ecotropic retroviruses with the constructs of interest. After determining viral titers by infecting NIH3T3 cells, the in vitro immortalizing capabilities of the fusion constructs in murine bone marrow progenitor cells will be determined in serial replating assays modeled after those described by Lavau. Donor mice will be treated with 5-fluorouracil for marrow stimulation. After the mice are sacrificed, hematopoietic bone marrow progenitor cells will be enriched for by depletion of cells expressing lineage differentiation markers. The bone marrow progenitor cells will be transduced with the retroviral supernatants by spinoculation. Presence of the desired sequence in the vector will be verified by RT-PCR prior to transduction and during the serial replating assay. The infected cells will be plated and incubated for ˜10 days in methylcellulose medium containing IL-3, IL-6, GM-CSF and SCF with or without G418. To assay immortalization, after the initial G418 selection, secondary, tertiary, and quaternary colonies will be cultured in the same medium without G418. In vitro immortalization of primary hematopoietic cells by the fusion constructs will be an indication of potential transforming properties of the fusion protein, which can be examined further in in vivo retroviral transplantation murine models. If the cells become immortalized in vitro, oligonucleotide arrays will be used to identify the gene expression profiles at each serial replating step. It is possible that a second genetic alteration is necessary for malignant transformation. The requirement for a putative secondary alteration will be tested via mutagenesis (e.g., retroviral mediated insertional mutagenesis, exposure to carcinogen etc.) in order to introduce additional alterations besides the translocations. If immortalization is seen after retroviral mutagenesis, the retroviral insertion sites will be identified by panhandle PCR to identify the genes involved in the alterations.

### DEFINITIONS

As used herein, the following terms have the meanings described in the present application.

The “bcr” region of MLL means the breakpoint cluster region of the MLL gene, an approximately 8.3-kilobase region of the gene which extends from a BamHI cleavage site of the sense strand of MLL exon 5 to another BamHI cleavage site of the sense strand of MLL exon 11. The sequence of the bcr of MLL is known (GenBank Accession # HSU04737). Where nucleotide residues are numbered within the bcr, they are numbered from the 5′-end of the sense strand of the bcr of MLL. Where breakpoints are identified within the bcr of MLL, the location of the breakpoint refers to the nucleotide residue located immediately 5′ of the site of breakage (i.e. the 3′-most residue of wild type MLL sequence following the translocation event).

A first region of a polynucleotide “flanks” a second region of the polynucleotide if the two regions are adjacent to one another, or if the two regions are separated by no more than about 1000 nucleotide residues, and preferably by no more than about 100 nucleotide residues.

A first region of a polynucleotide is “adjacent” to a second region of the polynucleotide if the two regions are attached to or positioned next to one another, having no intervening nucleotides. By way of example, the pentanucleotide region 5′-AAAAA-3′ is adjacent to the trinucleotide region 5′-TTT-3′ when the two are connected thus: 5′-AAAAATTT-3′ or 5′-TTTAAAAA-3′, but not when the two are connected thus: 5′-AAAAACTTT-3′.

“Complementary” refers to the broad concept of subunit sequence complementarity between regions of two polynucleotides or between two regions of the same polynucleotide. It is known that an adenine residue of a first polynucleotide region is capable of forming specific hydrogen bonds (“base pairing”) with a residue of a second polynucleotide region which is antiparallel to the first region if the residue is thymine or uracil. Similarly, it is known that a cytosine residue of a first polynucleotide region is capable of base pairing with a residue of a second polynucleotide region which is antiparallel to the first region if the residue is guanine. A first region of a polynucleotide is complementary to a second region of the same or a different polynucleotide if, when the two regions are arranged in an antiparallel fashion, at least three nucleotide residues of the first region is capable of base pairing with three residues of the second region. Preferably, the first region comprises a first portion and the second region comprises a second portion, whereby, when the first and second portions are arranged in an antiparallel fashion, at least about 30%, and preferably at least about 75%, at least about 90%, or at least about 95% of the nucleotide residues of the first portion are capable of base pairing with nucleotide residues in the second portion. Such portions are said to exhibit 30%, 75%, 90%, and 95% complementarity, respectively. More preferably, all nucleotide residues of the first portion are capable of base pairing with nucleotide residues in the second portion (i.e. the first and second portions exhibit 100% complementarity).

A first polynucleotide region and a second polynucleotide region are “arranged in an antiparallel fashion” if, when the first region is fixed in space and extends in a direction from its 5′-end to its 3′-end, at least a portion of the second region lies parallel to the first region and extends in the same direction from its 3′-end to its 5′-end.

“Homologous” as used herein, refers to nucleotide sequence identity between two regions of the same polynucleotide or between regions of two different polynucleotides. When a nucleotide residue position in both regions is occupied by the same nucleotide residue, then the regions are homologous at that position. A first region is homologous to a second region if at least three nucleotide residue positions of each region are occupied by identical nucleotide residues. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ are 50% homologous. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. Such portions are said to exhibit 50%, 75%, 90%, and 95% homology, respectively More preferably, all nucleotide residue positions of each of the portions are occupied by the same nucleotide residue (i.e. the first and second portions exhibit 100% homologous).

A “leukemia-associated DNA sequence” means a DNA sequence of a human patient wherein translocation of genomic DNA into the DNA sequence or rearrangement of the DNA sequence is associated with onset, continuation, or relapse of leukemia in the patient. Leukemia-associated DNA sequences include, but are not limited to, genes, such as MLL, which are associated with onset, continuation, or relapse of acute leukemia. It is understood that changes in a leukemia-associated DNA sequence, such as a chromosomal translocation for example, may occur in a preleukemia phase before leukemia is clinically detected (or may occur without the subsequent development of leukemia).

A “transformed host cell” refers to any host cell comprising a fusion transcript of the invention. Host cells may contain cDNA, gDNA, or RNA encoding the fusion proteins described herein. Such nucleic acids may be stably integrated into the host cell or expressed transiently. Methods for introducing heterologous nucleic acids into host cells are well known in the art. See Ausubel et al.

A “region” and a “portion” of a polynucleotide are used interchangeably to mean a plurality of sequential nucleotide residues of the polynucleotide.

A “polynucleotide” means a single strand or parallel and anti-parallel strands of a nucleic acid. Thus, a polynucleotide may be either a single-stranded or a double-stranded nucleic acid.

An “oligonucleotide” means a nucleic acid comprising at least two nucleotide residues.

A first polynucleotide is “ligated” to a second polynucleotide if an end of the first polynucleotide is covalently bonded to an end of the second polynucleotide. By way of example, the covalent bond may be a phosphodiester bond. “Extending” a polynucleotide means the addition of nucleotide residues to an end of the polynucleotide, wherein the added nucleotide residues are complementary to nucleotide residues of a region of either the same or a different polynucleotide with which the polynucleotide is annealed. Extension of a polynucleotide typically occurs by template-directed polymerization or by template-directed ligation.

A first polynucleotide is “annealed” with a second polynucleotide when the two polynucleotides are arranged in an anti-parallel fashion and when at least three nucleotide residues of the first polynucleotide are base paired with a nucleotide residue of the second polynucleotide.

A “panhandle structure” is a polynucleotide comprising a first region and a second region, wherein when the first region and the second region are separated by at least several nucleotide residues and are annealed to each other in an anti-parallel fashion. The first and second regions may be separated by several hundred or even by several thousand nucleotide residues.

A “primer” is an oligonucleotide which can be extended when annealed with a complementary region of a nucleic acid strand.

“Amplification of a region of a polynucleotide” means production of a plurality of nucleic acid strands comprising the region.

A “product” of an amplification reaction such as PCR means a polynucleotide generated by extension of a primer used in the amplification reaction.

A first polynucleotide comprises an “overhanging region” if it has a double-stranded portion wherein either the 3′-end or the 5′-end of a strand of the polynucleotide extends beyond the 5′-end or the 3′-end, respectively, of the same or a different strand of the polynucleotide. By way of example, the 5′-end of an antisense strand overhangs the 3′-end of a sense strand with which it is annealed if the 5′-end of the antisense strand extends beyond the 3′-end of the sense strand.

A “genomic DNA” of a human patient is a DNA strand which has a nucleotide sequence homologous with or complementary to a portion of a chromosome of the patient. Included in this definition for the purposes of simplicity are both a fragment of a chromosome and a cDNA derived by reverse transcription of a human RNA.

A “translocation partner” of a human gene is a region of genomic DNA which does not normally flank the gene, but which flanks the gene following a translocation event.

A “translocation event” means fusion of a first region of a human chromosome with a second region of a human chromosome, wherein the first region and the second region are not normally fused. By way of example, breakage of a first and a second human chromosome and fusion of a part of the first chromosome with a part of the second chromosome is a translocation event. For the sake of simplicity, tandem duplications are herein included within the definition of translocation event, it being understood that tandem duplications and translocations occur by similar mechanisms of DNA recombination.

A polynucleotide is “derived from” a gene if the polynucleotide has a nucleotide sequence which is either homologous with or complementary to a portion of the nucleotide sequence of the gene.

A first polynucleotide anneals with a second polynucleotide “with high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g. Sambrook et al., 1989, Molecular Cloning: A Laboratory Manual, Cold Spring Harbor Laboratory, New York).

A third primer is “nested” with respect to a first primer and a second primer if amplification of a region of a first polynucleotide using the first primer and the second primer yields a second polynucleotide, wherein the third primer is complementary to an internal portion of the second polynucleotide, wherein the internal portion of the second polynucleotide to which it is complementary does not include a nucleotide residue at the corresponding end of the second polynucleotide.

A second primer is “nested” with respect to a first primer if amplification of a region of a first polynucleotide using the first primer yields a second polynucleotide, wherein the second primer is complementary to an internal portion of the second polynucleotide, and wherein the internal portion of the second polynucleotide to which the second primer is complementary does not include a nucleotide residue at the corresponding end of the second polynucleotide.

A portion of a polynucleotide is “near” the end of a region of the polynucleotide if at least one nucleotide residue of the portion is separated from the end of the region by no more than about one hundred nucleotide residues, and preferably by no more than about twenty-five nucleotide residues.

A restriction site is a portion of a polynucleotide which is recognized by a restriction endonuclease. A portion of a polynucleotide is “recognized” by a restriction endonuclease if the endonuclease is capable of cleaving a strand of the polynucleotide at a fixed position with respect to the portion of the polynucleotide.

A strand of a polynucleotide is the “sense” strand with respect to unknown flanking DNA if the nucleotide sequence of a first portion of the strand is known, if the nucleotide sequence of a second portion of the strand is unknown, and if the first portion is located 5′ with respect to the second portion.

The following examples are provided to illustrate certain embodiments of the invention. They are not intended to limit the invention in any way.

### EXAMPLE I

Identification of New Translocation Partner for MLL

This study focuses on a patient who has been treated for neuroblastoma and developed a MLL translocation, but has yet to develop leukemia. During chemotherapy, a bone marrow karyotype revealed a t(4;11) MLL translocation involving 11q23 and 4p12. 11q23 is the MLL gene, but a chromosome 4 translocation usually involves, AF-4, located at 4q21. The location of this partner gene was novel and was newly named MIFL (mixed-lineage leukemia insufficient for leukemia) using a new panhandle technique, BglII reverse panhandle PCR. MIFL codes for the KIAA0826 protein, but the functions of this partner gene and protein have not been explored and are uncharacterized.

The patient has been in remission for over 2 years, finishing the N8 regimen chemotherapy treatment in August 2003. Yet the MLL translocation identified has been present ever since the end of treatment (May 2003). In addition to determining the der(11) and der(4) genomic breakpoint junctions, fusion transcripts involving both genes have been discovered using RT-PCR with clonotypic cDNA primers. The function and/or the abundance of these transcripts and their protein products may shed light on the reason(s) this patient has not developed leukemia. MLL itself is involved in transcriptional regulation and epigenetic control of other genes, including those involved in hematopoiesis, and it is possible that the translocation disrupts the normal function of the gene product of either MLL itself or the partner gene or both. Real-time PCR has also been utilized to show the differences in gene expression of this patient over a variety of common leukemic genes, such as the family members of HOXA and co-factors, and between other de novo or treatment-related AML and ALL leukemia specimens from patients. BglII reverse panhandle PCR is a powerful technique and may be able to aid other patient samples that cannot be cloned using traditional BamHI-digested DNA with panhandle or reverse panhandle PCR.

The following materials and methods are provided to facilitate the practice of Example I.

Case History

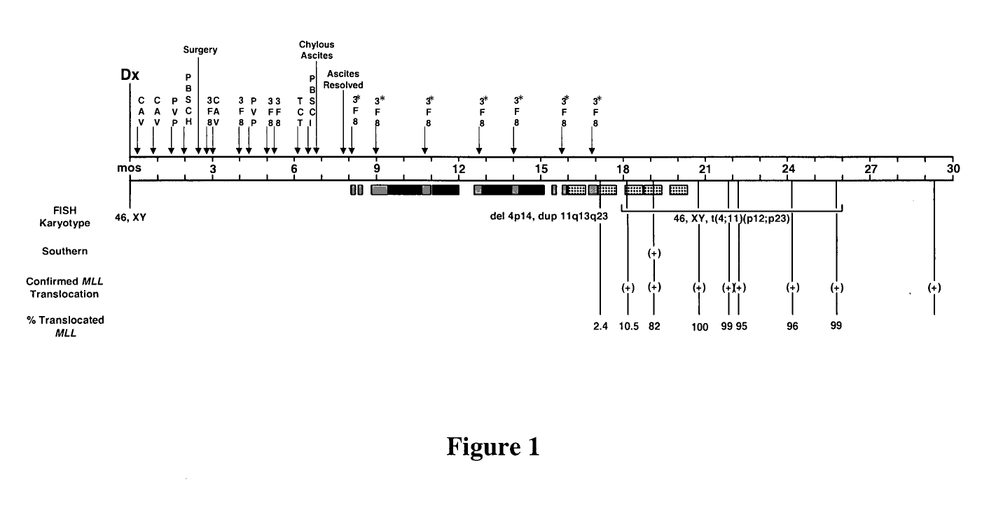

Patient NB142 was diagnosed with stage IV neuroblastoma at age 4.5 years. Treatment included intensive chemotherapy per the Memorial Sloan Kettering N8 regimen with 3 cycles of cyclophosphamide/doxorubicin/vincristine, 2 cycles of cisplatin/etoposide (PVP), surgery, local radiation, anti-G_{D2} monoclonal antibody (3F8), myeloablative therapy (thiotepa, carboplatin, topotecan) plus autologous PBSC transplant (harvested after PVP#1), followed by 7 more cycles of 3F8 plus GM-CSF, 4 cycles of oral VP, and 5 cycles of accutane. Cumulative doses of doxorubicin and VP were 225 mg/m² and 5.4 g/m² (4.2 g/m² as oral VP), respectively. About 18.5 months after initial diagnosis the patient was asymptomatic but examination of the marrow revealed a translocation involving MLL. The patient has not developed leukemia as of Jul. 12, 2004. The karyotype was 46, XY, t(4;11)(p12;q23) in 8 out of 21 metaphase cells examined initially in June of 2003, but is now found in greater than 95% of the cells (FIG. 1).

Detection of MLL Gene Rearrangements by Southern Blot Analysis

High molecular weight genomic DNA was isolated by ultracentrifugation on 4 M guanidine isothiocyante/5.7 M CsCl gradients (Felix et al. (1990) J. Clin. Oncol., 8:431-42). The 8.3-kb MLL bcr was examined by Southern blot analysis of BamHI, BamHI/BglII, and BglII digested DNAs using the B859 cDNA fragment of ALL-1 exons 5-11 (Gu et al. (1992) Cell, 71:701-708). The sizes of the rearrangements on the Southern blot are approximate sizes of the target sequences for PCR (Felix et al. (1997) Blood, 90:4679-86; Megonigal et al. (1997) Proc. Natl. Acad. Sci., 94:11583-8).

Characterization of der(4) Genomic Breakpoint Junction using BglII Reverse Panhandle Polymerase Chain Reaction

For patient NB142, BglII digested DNA and adapted reverse panhandle PCR was used to amplify the breakpoint from a der(4) chromosome with known 3′ MLL sequence juxtaposed to 5′ unknown partner. Genomic DNA was digested at 37° C. for 2 hours with BglII to create a restriction fragment with a 5′ overhang. The 100-μl reaction contained 5 μg of genomic DNA, 20 units of BglII, and 10× React 3 buffer (Invitrogen, Carlsbad, Calif.). 0.05 units of calf intestinal alkaline phosphatase (Boehringer Mannheim Biochemicals, Indianapolis, Ind.) was added to the digested DNA and incubated at 37° C. for 30 minutes. The digested phosphatase-treated DNA was purified by using a Geneclean III kit (Bio 101, La Jolla, Calif.) and resuspended in 25 μl dH₂O. A single-stranded 5′ phosphorylated oligonucleotide that was homologous to known MLL sequence at positions 8004-8035 in exon 10 (GenBank accession no. U04737) in the bcr was ligated to the 3′ ends of the BglII digested DNA at 4° C. overnight. The sequence was 5′-GAT CAC CCT GAG TGC CTG GGA CCA AAC TAC CCC ACC, wherein the underlined represented the BglII overhang. The 50-μl ligation reaction contained 16.9 μl of dH₂O, 25 μl of digested, phosphatase-treated DNA (5 μg), 2.1 μl of 5′ phosphorylated oligonucleotide (0.25 μg/μl), 5 μl of 10× ligase buffer, and 1 μl of T4 DNA ligase (1 unit/μl) (Boehringer Mannheim Biochemicals). The DNA was again purified using a Geneclean III kit (Bio 101) and resuspended in 25 μl dH₂O. Two microliters of digested, ligated DNA was added to a 43-μl mixture containing 1 μl of Elongase Enzyme Mix (1 unit/μl), 1 μl of 10 mM dNTP mix, 5 μl of 5× Buffer A, and 5 μl of 5× Buffer B (Elongase Amplification System, Invitrogen). The mixture was heated to 80° C. for 5 minutes before the DNA was added. The DNA was made single-stranded by heating the reaction mixture at 94° C. for 1 minute. The stem-loop template was formed by a 2 minute ramp to 72° C. and incubated at 72° C. for 30 seconds to promote intrastrand annealing of the ligated oligonucleotide to the complementary sequence in the antisense strand and polymerase extension of the recessed 3′ end (Felix et al. (1997) Blood, 90:4679-86). With the reaction mixture at 80° C., 12.5 pmoles of primer 1 corresponding to MLL intron 10 antisense positions 8089-8063 (5′-TAA AAG AGC ATC ATG TGT ATA ACT CAC-3′) and of primer 2 corresponding to MLL exon 10 antisense positions 8017-7991 (5′-CAG GCA CTC AGG GTG ATA GCT GTT TCG-3′) was added. Each final 50-μl PCR reaction mixture contained 12.5 pmol each of primers 1 and 2, 200 μM of each dNTP, and 1× PCR buffer. After denaturation at 94° C. for 1 minute, 10 cycles at 94° C. for 10 seconds and 68° C. for 7 minutes, and 20 cycles at 94° C. for 10 seconds and 68° C. for 7 minutes (increment, 20 seconds/cycle) were used, followed by final elongation at 68° C. for 7 minutes. Nested PCR was performed using 1 μl of the product of the preceding PCR as a template. The PCR product was added to a 49-μl mixture containing 1 μl of Elongase Enzyme Mix (1 unit/μl), 1 ul of 10 mM dNTP mix, 5 μl of 5× Buffer A, 5 μl of 5× Buffer B (Elongase Amplification System, Invitrogen), 12.5 pmoles of primer 3 corresponding to MLL exon 10 antisense positions 8062-8036 (5′-CCA GAC TTT CTT CTT CTT TGT GGG TTT-3′), and of primer 4 corresponding to MLL exon 10 antisense positions 8000-7971 (5′-AGC TGT TTC GGC ACT TAT TAC ACT CCA GCA-3′). The mixture was heated to 80° C. for 5 minutes before the PCR product was added. Each final 50-μl PCR reaction mixture contained 12.5 pmol each of primers 3 and 4, 200 μM of each dNTP, and 1× PCR buffer. Nested PCR conditions were the same as in the initial PCR.

The BglII reverse panhandle PCR products were subcloned into the pCR 2.1-TOPO vector using a TOPO TA Cloning Kit (Invitrogen). The nested PCR product was purified using a Geneclean III kit (Qbiogene; Carlsbad, Calif.) and resuspended in 10 μl dH₂O (Bio 101). Purified PCR product (2.5 μl) was added to a mixture containing 1 μl salt solution (1.2 M NaCl, 0.06 M MgCl₂), 1.5 μl dH₂O, and 1 μl TOPO vector (pCR 2.1-TOPO) and incubated at room temperature for 15 minutes. Two microliters of the TOPO cloning reaction was added to a vial of One Shot DH5α-T1 Chemically Competent Cells (Invitrogen). The reaction was incubated on ice for 30 minutes, followed by heat-shocking the cells for 30 seconds at 42° C. The heat-shocked cells were put on ice for 2 minutes prior to adding 300 μl S.O.C. medium (Invitrogen) and allowed to recover at 37° C. for 1 hour with shaking (225 rpm). 50 or 300 μl of the transformation reaction was plated on LB/agar plates containing carbenicillin (100 μg/ml) and incubated overnight at 37° C. Individual transformants were grown up for 5 hours at 37° C. in 2 ml of LB broth containing carbenicillin (100 μg/ml).

PCR was used to identify recombinant plasmids containing products of BglII reverse panhandle PCR. Two microliters of the saturated 2 ml cultures were amplified in PCRs containing 1 μl of Elongase Enzyme Mix (1 unit/μl), 1 μl of 10 mM dNTP mix, 5 μl of 5× Buffer A, 5 μl of 5× Buffer B (Elongase Amplification System, Invitrogen), 12.5 pmoles of primer 3 corresponding to MLL exon 10 antisense positions 8062-8036 (5′- CCA GAC TTT CTT CTT CTT TGT GGG TTT-3′), and of primer 4 corresponding to MLL exon 10 antisense positions 8000-7971 (5′-AGC TGT TTC GGC ACT TAT TAC ACT CCA GCA-3′). Each final 50-μl PCR screen reaction mixture contained 12.5 pmol each of primers 3 and 4, 200 μM of each dNTP, and 1× PCR buffer. PCR screen conditions were the same as in the initial PCR. Agarose gel electrophoresis of the products identified transformants containing the recombinant plasmid DNA of interest, which then were grown in 4-ml cultures for plasmid preparation and automated sequencing.

Validation of der(4) and Identification of der(11) Genomic Translocation Breakpoints by Conventional PCR and Direct Genomic Sequencing

To detect the der(4) and der(11) translocation breakpoints by an independent method, we amplified fresh aliquots of genomic DNA from patient NB142 with primers encompassing the known der(4) and the predicted der(11) translocation breakpoints, which were designed from sequences of the products of BglII reverse panhandle PCR. Forward and reverse PCR primers derived for the der(4) translocation breakpoint were 5′-GCT GAA ACC ATG GAA GGA AA-3′ (corresponding to positions 1149-1168 in intron 8 of the gene corresponding to KIAA0826 (Accession No. AB020633) and 5′-AAA AAT TCG CAT GGA GGA GA-3′ (corresponding to positions 5604-5585 in MLL bcr (accession No. U04737), respectively, and would yield a 358-bp product. Forward and reverse PCR primers derived for the der(11) translocation breakpoint were 5′-TCT ACA AGT GCC AGG GGT CT-3′ (corresponding to MLL bcr positions 5315 to 5334 (Accession No. U04737) and 5′-AAA GCC AAG CTT CTC CAT CA-3′ (corresponding to positions 1680-1661 (in intron 8) of the partner gene (Accession No. AB020633), respectively, and would yield a 464-bp product. Fifty nanograms of genomic DNA were amplified in 50-μl reaction mixtures containing 3.5 units of Taq/Tgo DNA polymerase (Expand High Fidelity PCR System, Roche), all four dNTPs (250 μM each), PCR buffer at 1× final concentration, and 12.5 pmoles of each primer. After initial denaturation at 94° C. for 9 minutes, 35 cycles at 94° C. for 1 minute, 55° C. for 1 minute, and 72° C. for 2 minutes were used, followed by final elongation at 72° C. for 7 minutes. Products of reactions were isolated from a 1.2% agarose gel with a Geneclean III kit (Bio 101) and resuspended in 20 μl dH₂O. Automated methods were used to sequence in both directions to validate and identify the breakpoints.

Tracking the der(4) and der(11) Breakpoints

DNA was isolated from marrows on glass slides by using Puregene DNA Isolation Kit for tissue samples (Gentra Systems, Minneapolis, Minn.) according to the manufacturer's directions. Forward and reverse initial PCR primers derived for the der(4) translocation breakpoint were 5′-GCT GAA ACC ATG GAA GGA AA-3′ and 5′-CTC CCA AAG TGC TGG GAT TA-3′, respectively, and would yield a 870-bp product. Forward and reverse initial PCR primers derived for the der(11) translocation breakpoint were 5′-GCT GCA GTG AGC CAT TAT CA-3′ (corresponding to MLL bcr positions 4997 to 5016 in intron 8) and 5′-AAA GCC AAG CTT CTC CAT CA-3′ (corresponding to positions 1680 to 1661 of intron 8 of the partner gene), respectively, and would yield a 738-bp product. Forward and reverse nested PCR primers derived for the der(4) translocation breakpoint were 5′-GCT GAA ACC ATG GAA GGA AA-3′ and 5′-AAA AAT TCG CAT GGA GGA GA-3′, respectively, and would yield a 358-bp product. Forward and reverse nested PCR primers derived for the der(11) translocation breakpoint were 5′-TCT ACA AGT GCC AGG GGT CT-3′ (corresponding to MLL bcr positions 5315 to 5334 in intron 8) and 5′-AAA GCC AAG CTT CTC CAT CA-3′, respectively, and would yield a 464-bp product. One hundred nanograms of genomic DNA were amplified in 50-μl reaction mixtures containing 3.5 units of Taq/Tgo DNA polymerase (Expand High Fidelity PCR System, Roche), all four dNTPs (250 μM each), PCR buffer at 1× final concentration, and 12.5 pmoles of each primer. After initial denaturation at 94° C. for 9 minutes, 35 cycles at 94° C. for 1 minute, 55° C. for 1 minute, and 72° C. for 2 minutes were used, followed by final elongation at 72° C. for 7 minutes. Nested PCR was performed using 1 μl of the initial PCR products with the same reaction mixture and PCR conditions. Nested PCR products of reactions were visualized on a 2% agarose gel to verify breakpoint. Some samples were isolated from a 2% agarose gel with a Geneclean III kit (Bio 101) and resuspended in 20 μl dH₂O. Automated methods were used to sequence some samples in both directions to validate the breakpoints. Serial 1:10 dilutions of marrow DNA into peripheral blood lymphocyte DNA from a normal subject were analyzed to determine the sensitivity of the assay.

Reverse Transcriptase-PCR Analysis of Fusion Transcripts

RT-PCR analysis was performed to evaluate whether the translocation generated a MLL-KIAA0826 chimeric mRNA. First-strand cDNA was synthesized from 10 μg of total RNA from patient NB142 with random hexamers using the High Capacity cDNA Archive Kit (Applied Biosystems) according to the manufacturer's directions. The 50-μl RT-PCR mixtures contained 2 μl of random hexamer-primed cDNA (100 ng), 2.6 units of Taq/Tgo DNA polymerase (Expand High Fidelity PCR System, Roche; Indianapolis, Ind.), all four dNTPs (200 μM each), PCR buffer at 1× final concentration, and 12.5 pmoles of each primer. The der(4) transcript of patient NB142 was identified by amplification of random hexamer-primed cDNA with the KIAA0826 exon 8 (GenBank accession number AB020633) sense primer 5′-CTG GAA GGA AGC CCT TAA CA-3′ (from positions 1383-1402) and the MLL exon 10 (GenBank accession number L04284) antisense primer 5′-TTT GGT GGG GTA GTT TGG TC-3′ (from positions 4551-4532). The der(11) transcripts of patient NB142 were identified by amplification of random hexamer-primed cDNA with the MLL exon 7 sense primer 5′- GAA TGC AGG CAC TTT GAA CA-3′ (corresponding to positions 4116-4135 of the full length MLL cDNA (Accession No. L04284) and the KIAA0826 exon 11 antisense primer 5′- TCA CCG ACT TCC AAA TCC TC-3′ (corresponding to positions 1847-1828 in partner gene cDNA (KIAA0826). After initial denaturation at 94° C. for 9 minutes, 35 cycles at 94° C. for 1 minute, 55° C. for 1 minute, and 72° C. for 2 minutes were used, followed by final elongation at 72° C. for 7 minutes. Products of reactions were isolated from a 2% agarose gel with a Geneclean III kit (Qbiogene) and resuspended in 20 μl dH₂O. Automated methods were used to sequence in both directions to identify the fusion transcripts.

Taqman (Real-Time Quantitative PCR)

First-strand cDNA was synthesized from 1-10 μg of total RNA from patients p38, p45, p96, t-120, t-126, t-127, and NB142 with random hexamers using the High Capacity cDNA Archive Kit (Applied Biosystems) according to the manufacturer's directions. RQ-PCR was carried out using TaqMan probe-based chemistry (Applied Biosystems). All gene-specific primers and probes were purchased as Assay-On-Demand kits (Applied Biosystems). Target genes of interest were members of the HOX gene family (HOXA4, HOXA5, HOXA7, HOXA9), HOX co-factors (Meis1, Pbx3), Flt3, and Bcl2. 18S rRNA was the endogenous control and was purchased in a Pre Developed Assay Reagent kit (Applied Biosystems). The amplification reactions (20.0 μL) contained 100 ng cDNA from patient samples, 10 μl 2× Taqman Universal Master Mix, and 1 μl 20× endogenous control (18S rRNA) or 20× target gene. Amplifications were performed following an initial 2-minute incubation at 50° C. to allow uracil-N-glycosylase (UNG) to destroy any contaminating RNA, followed by treatment at 95° C. for 10 minutes to inactivate the UNG enzyme and activate the AmpliTaq Gold DNA polymerase. This was followed by 50 cycles of denaturing at 95° C. for 15 seconds and annealing/extension at 60° C. for 1 minute. An ABI Prism 7900HT Sequence Detection System equipped with a 384-well thermal cycler was used for the amplifications. Data were collected and analyzed with SDS 2.1 software (Applied Biosystems). Standard curves were used (2 μg to 1 ng) to aid in absolute quantification of the results. The comparative C_{T} method was used for relative quantification of gene expression.

Bone Marrow Slide DNA Extraction

DNA was extracted from bone marrow glass slides using a Puregene DNA Isolation Kit (Gentra Systems, Minneapolis, Minn.). For each slide, 300 μl lysis buffer and 1.5 μl proteinase K (20 mg/ml) were mixed in a 1.5 ml microcentrifuge tube. Fifteen microliters of this mixture was added to a glass slide containing bone marrow. The bone marrow was scraped off into the microcentrifuge tube containing the lysis buffer and proteinase K. The tubes were inverted 25 times and incubated overnight at 55° C. in a heating block to dissolve the bone marrow. Once dissolved, 1.5 μl RNase A (4 mg/ml) was added to the tube, inverted 25 times, and incubated at 37° C. for 60 minutes. The tubes were briefly centrifuged and cooled to room temperature before adding 100 μl protein precipitation solution. The tubes were vortexed for 20 seconds and then incubated at 4° C. for 10 minutes. Following incubation, the tubes were centrifuged at 14000 rpm for 5 minutes prior to adding the supernatant to a new microcentrifuge tube containing 300 μl isopropanol and 0.5 μl glycogen (20 mg/ml). The tubes were inverted 50 times and then incubated at −20° C. for 60 minutes. Tubes were centrifuged for 5 minutes at 14000 rpm, the supernatant was discarded, and the tubes were allowed to air dry for 5 minutes. Once dry, 300 μl 70% ethanol was added and incubated at −20° C. for 30 minutes. The tubes were then centrifuged for 5 minutes at 14000 rpm and the supernatant was discarded. The pellet was allowed to air dry for 15 minutes, then 50 μl hydration solution (TE) was added, the tubes were incubated at 65° C. for 60 minutes, and then allowed to rehydrate at room temperature overnight before determining the concentration of DNA.

Preparation of Singly 5′ End-Labeled Substrates.

Fragments of the MLL bcr spanning positions 5382-5570 in intron 8 or fragments of MIFL spanning positions 1309-1479 in intron 8 were subcloned into BamHI-NotI sites of TOPO pCR2.1 (Invitrogen). Twenty micrograms of plasmid DNA first was treated with 400 units T4 DNA ligase (New England Biolabs; Beverly, Mass.) for 1 hour to ensure that the substrates were not nicked. Following extraction with 25:24:1 phenol:chloroform:isoamyl alcohol (PCI) and ethanol precipitation, plasmids were digested with 40 units of BamHI (Invitrogen) and 45 units of NotI (Invitrogen) followed by dephosphorylation with 20 units of calf intestinal phosphatase (New England Biolabs). Calf intestinal phosphatase was inactivated in 5 mM EDTA (pH 8.0) and the DNA was PCI-extracted and ethanol precipitated. The DNA was then 5′ end-labeled with [γ-³²P]ATP (Amersham; Arlington Heights, Ill.) and T4 polynucleotide kinase (New England Biolabs) as described (Felix et al. (1995) Cancer Res., 55:4287-92). Alternatively, to prepare cold substrate DNA, 20 μg of BamHI/NotI-digested, dephosphorylated plasmid were phosphorylated with 10 μM ATP (Boehringer Mannheim; Indianapolis, Ind.) in otherwise similar reactions. After heat inactivation of the kinase at 70° C. for 20 min, the DNA was digested with 80 units of SpeI (Promega) to generate either singly 5′ end-labeled or cold BamHI-SpeI fragments containing the normal homologues of MLL translocation breakpoints. The DNA fragments were resolved in 1.5% agarose gels, excised upon visualization with ethidium bromide staining, and cleaned using Geneclean III kit (Bio 101). DNA recovery was quantitated by optical density measurement and scintillation counting.

DNA Topoisomerase II in Vitro Cleavage Assays.

A total of 25 ng of substrate DNA containing 30,000 cpm labeled DNA and the remainder cold DNA were incubated at 37° C. for 10 min in 50 μL reactions containing 147 nM human DNA topoisomerase IIR, 1 mM ATP, 137.5 mM KCl, 12.5 mM Tris, pH 7.6, 5 MM MgCl₂, 105 μM EDTA, 25 μM dithiothreitol, 4.5% (v/v) glycerol, 8% (v/v) DMSO, and either 20 μM final concentrations of etoposide, etoposide catechol, etoposide quinone, or doxirubicin. Additional reactions were performed in the absence of drug or DNA topoisomerase II. Covalent complexes were irreversibly trapped by adding 5 μL of 10% SDS without or with prior incubation for 10 min at 75° C., the latter to evaluate the heat stability of the complexes. Following addition of 3.75 μL of 250 mM EDTA, cleavage products were deproteinized by incubation with 5.0 μL of 0.8 mg/ml proteinase K (Amersham) for 30 minutes at 45° C. Twenty micrograms of yeast tRNA was added, and the cleavage products were ethanol precipitated twice, resuspended in 6 μL of Sequenase Stop Solution (Amersham), and resolved in an 8% polyacrylamide-7.0 M urea gel in parallel with dideoxy sequencing reactions primed at the same 5′ end. Cleavage products were visualized by autoradiography and quantitated using a PhosphorImager and ImageQuant software (Molecular Dynamics; Sunnyvale, Calif.).

Results

Characterization of der(4) and der(11) Genomic Breakpoint Junctions and Fusion Transcripts From t(4;11)

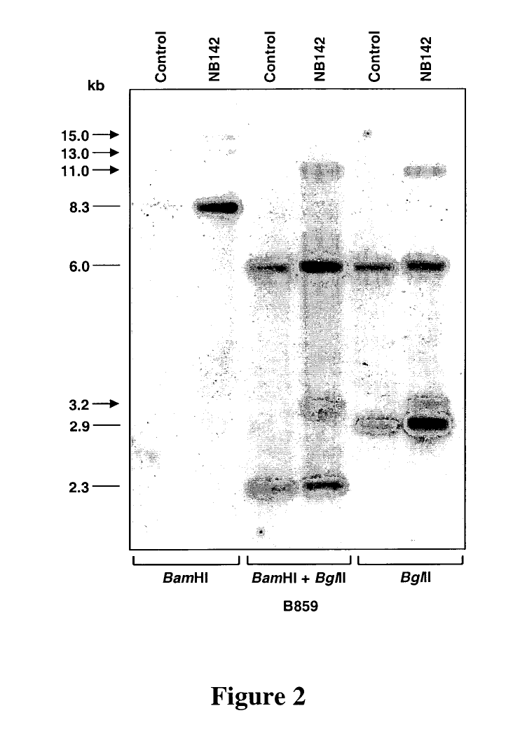

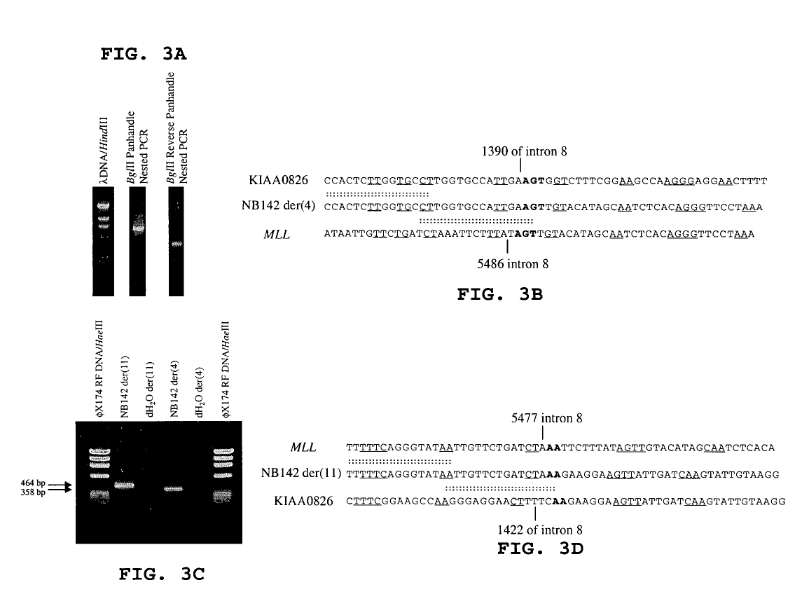

The reciprocal-genomic breakpoint junctions and resulting fusion transcripts from the translocation were characterized in cryopreserved bone marrow cells obtained from patient NB142 19 months after neuroblastoma diagnosis, in which 82% of the cells contained the translocation. Southern blot analysis of BamHI-digested DNA of patient NB142 revealed 15.0 and 13.0 kb rearrangements, which are too large to clone using traditional panhandle or reverse panhandle PCR methods (FIG. 2). Southern blot analysis of BglII-digested DNA (and BamHI/BglII digested DNA) of patient NB142 revealed 11.0 kb and 3.2 kb MLL bcr rearrangements (FIG. 2). There previously had been no known MLL partner genes at chromosome band 4p12. BLAST query of the partner gene sequence in the BglII reverse panhandle PCR product returned a sequence with 100% identity within chromosome band 4p12 (GenBank accession no. AB020633). Thus, BglII Panhandle and Reverse Panhandle PCR were used to determine which rearrangement was the der(11) or der(other). BglII Panhandle PCR could only amplify the 6.0 kb germline fragment, but BglII Reverse Panhandle PCR was able to amplify the der(4) genomic breakpoint junction (FIG. 3A). The 2947 bp product suggested that the 11.0 kb and 3.2 kb rearrangements on the Southern blot were from the der(11) and der(4) chromosomes, respectively (FIG. 2). Sequencing of TOPO TA cloning generated subclones which revealed the MLL der(4) breakpoint at position 5486, 5487, 5488, or 5489 in intron 8 (GenBank accession number U04737), also 3′ in the bcr, and the der(4) breakpoint in the unknown partner gene, that codes for the KIAA0826 (mRNA transcript) protein, position 1387, 1388, 1389, or 1389 in intron 8 (FIG. 3B). The MLL and KIAA0826 breakpoints could not be localized precisely because both genes contain an A, G, and T residue in the sequence at the breakpoint. Other 1-4-base homologies were present near the breakpoints in both genes (FIG. 3). Sequencing of the 358-bp product from PCR performed with KIAA0826 and MLL clonotypic primers confirmed the der(4) genomic breakpoint junction (FIG. 3C).

Clonotypic PCR was used to clone the der(11) genomic breakpoint junction due to the large size of the 11.0 kb rearrangement. Based on the der(4) genomic breakpoint cloned using BglII reverse panhandle PCR, clonotypic primers were designed surrounding the genomic breakpoint. Sequencing of the 464-bp product from PCR performed with MLL and KIAA0826 clonotypic primers identified the der(11) genomic breakpoint (FIG. 3C). The unknown partner gene encoding the KIAA0826 protein der(11) breakpoint was position 1422, 1423, or 1424 of intron 8; the MLL der(11) breakpoint was position 5475, 5476, or 5477 in intron 8 (FIG. 3D). In addition to 5′-AA-3′ immediately at the breakpoints in both genes that precluded more precise assignments, other short homologous sequences in MLL and KIAA0826 flanked the der(11) genomic breakpoint junction (FIG. 3D). Several bases from MLL and KIAA0826 were lost during the translocation process. Depending on the exact positions of the der(11) and der(4) MLL and KIAA0826 genomic breakpoints, the deleted region from MLL intron 8 was 8-13 bp and the deleted region from KIAA0826 intron 8 was 31-36 bp.

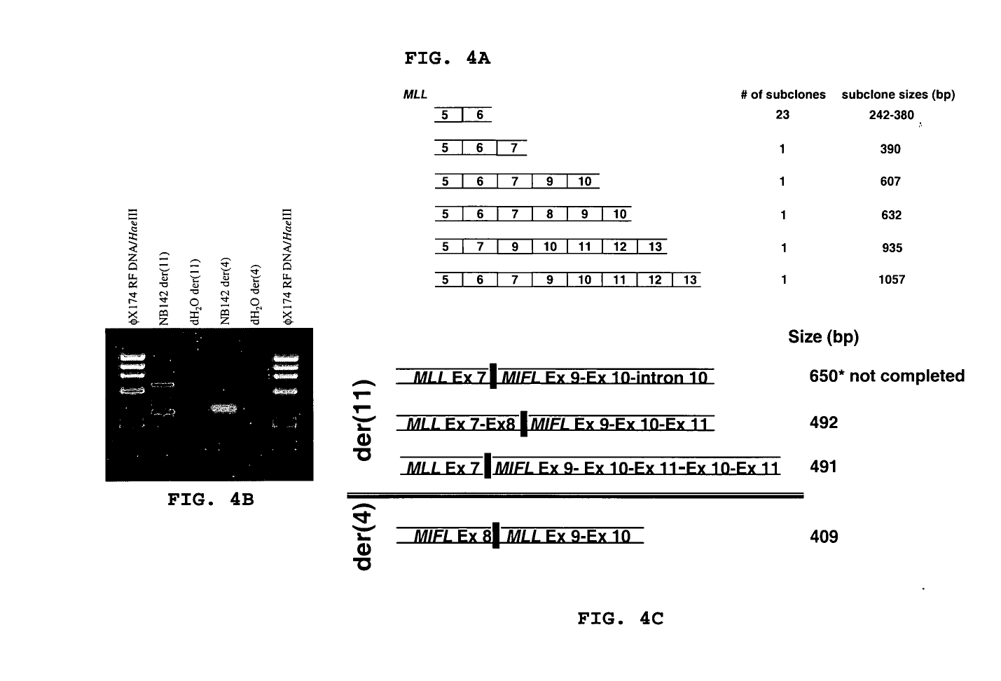

cDNA panhandle PCR did not identify any in-frame chimeric transcripts from the der(11) chromosome joining MLL to KIAA0826 from PCR-generated subclones that were sequenced, only transcripts involving MLL were identified (FIG. 4A). RT-PCR with MLL and KIAA0826-specific primers and sequencing of 3 PCR products (441-592 bp) identified 3 fusion transcripts (FIG. 4B); the sequences indicated that the der(11) transcripts fused MLL exon 7 or 8 in-frame to KIAA0826 exon 9 (FIG. 4C). RT-PCR with KIAA0826 and MLL-specific primers gave a 363 bp product; the sequence indicated that the der(4) transcript fused KIAA0826 exon 8 in-frame to MLL exon 9 (FIG. 4C).

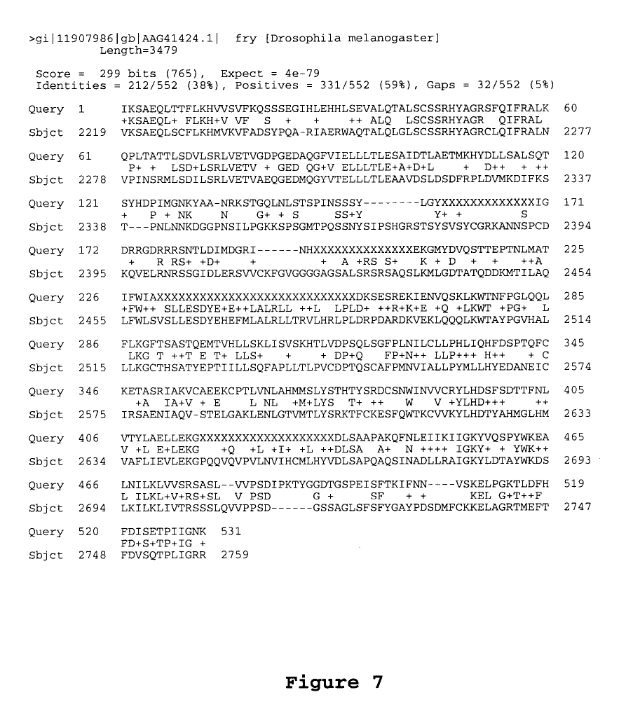

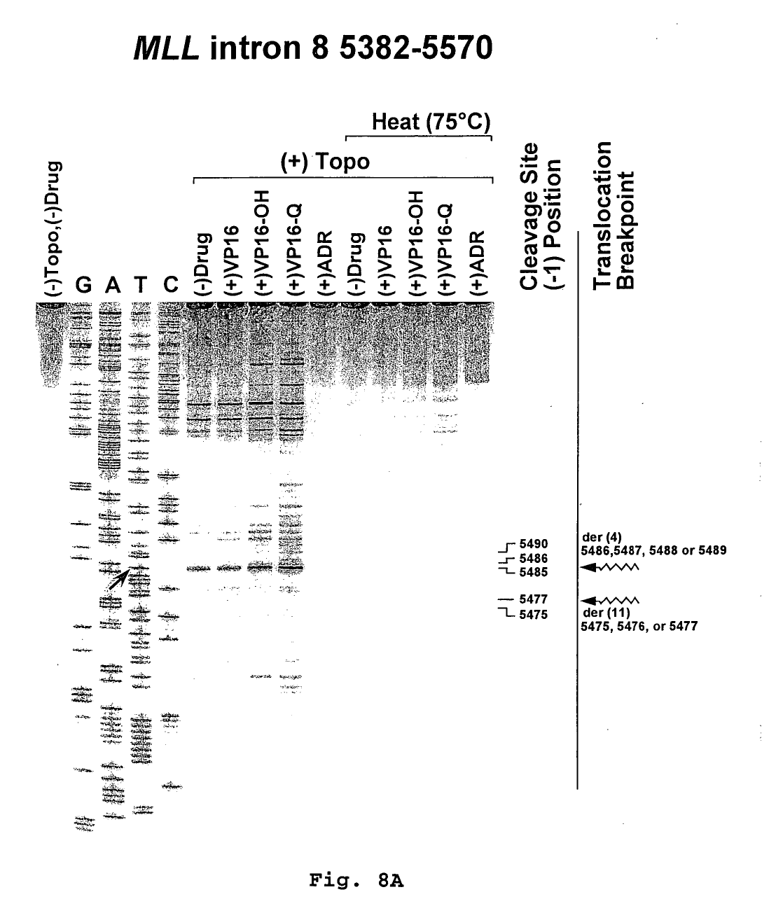

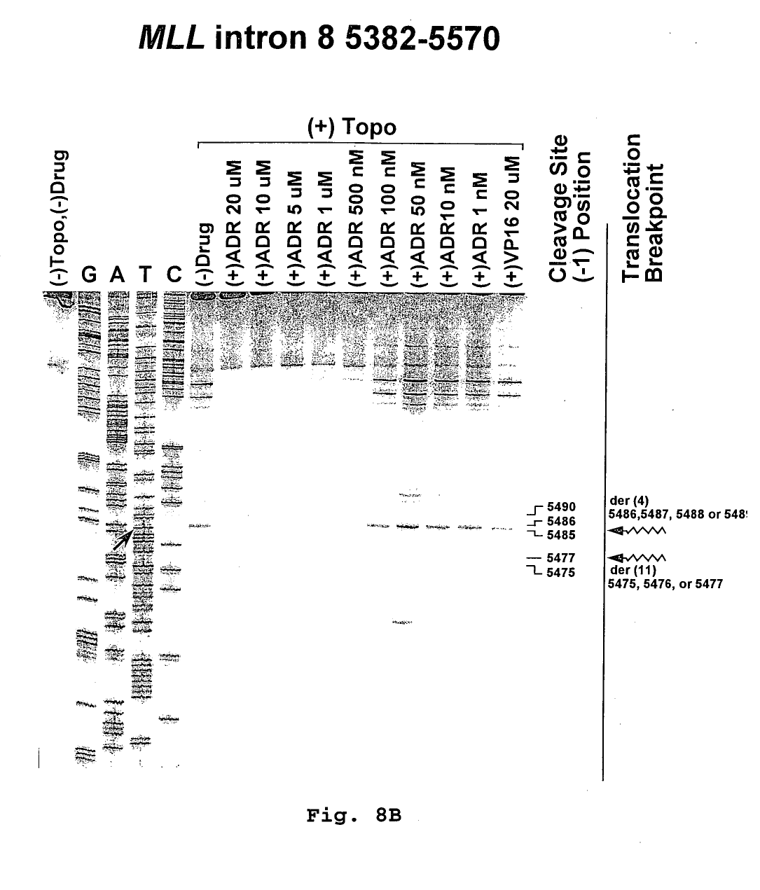

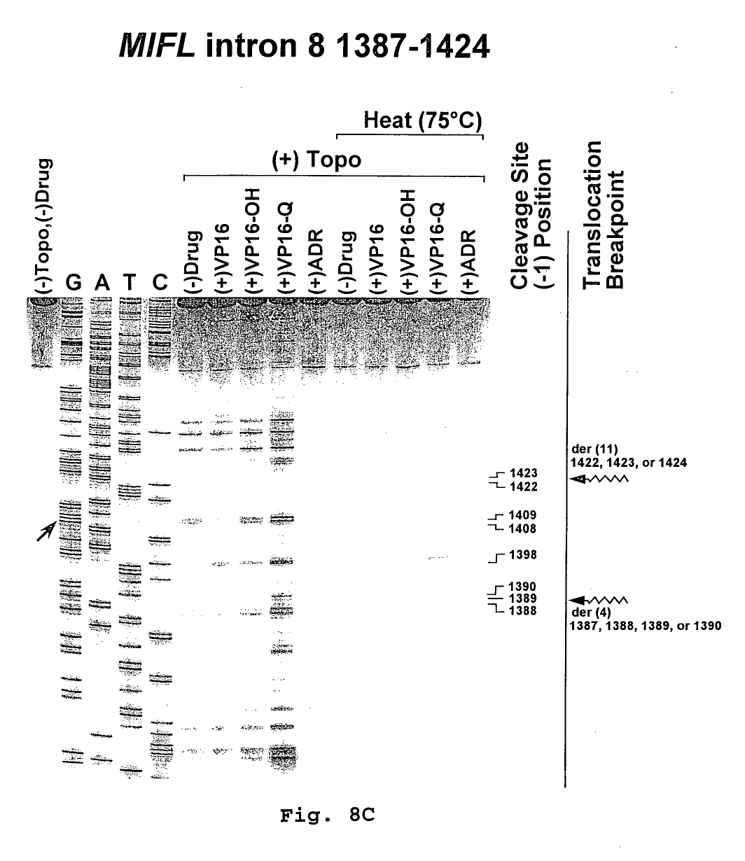

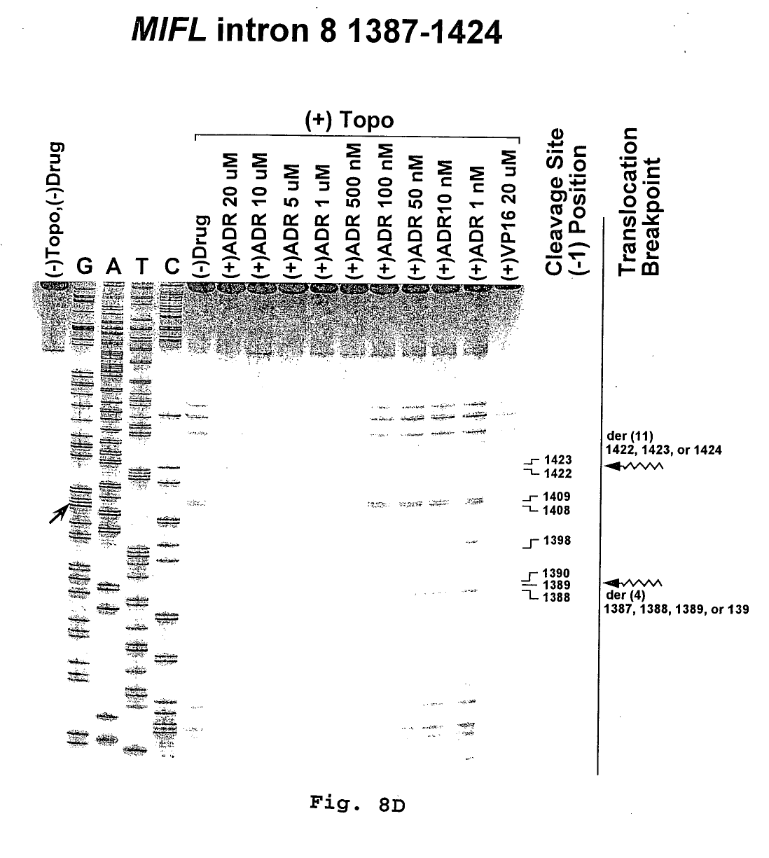

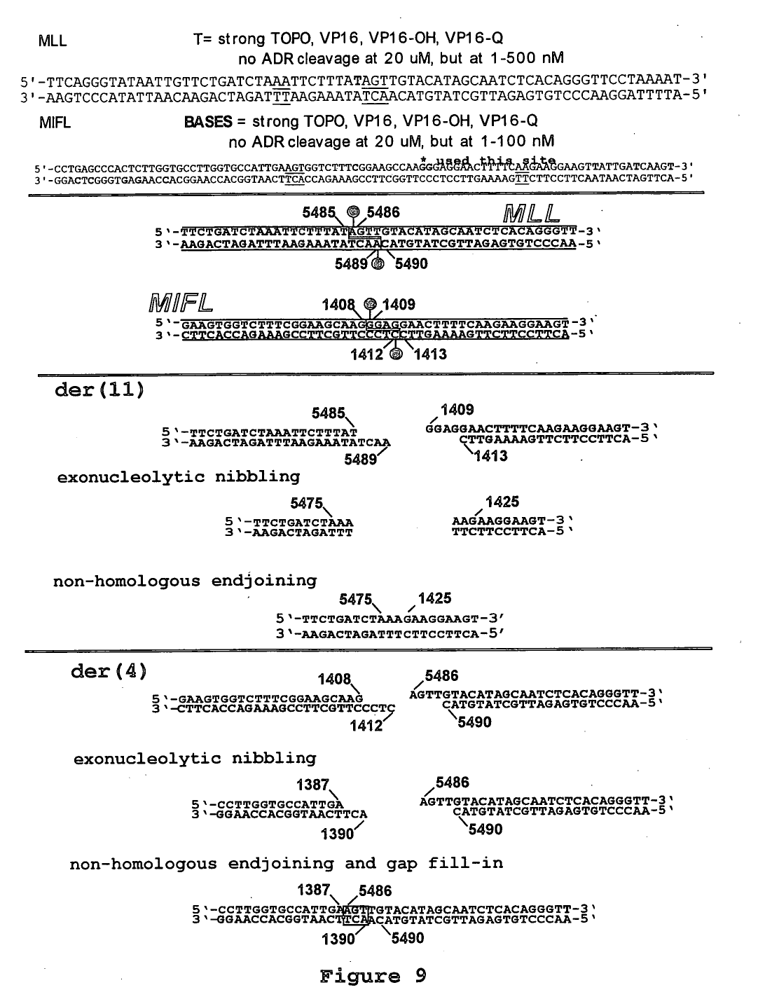

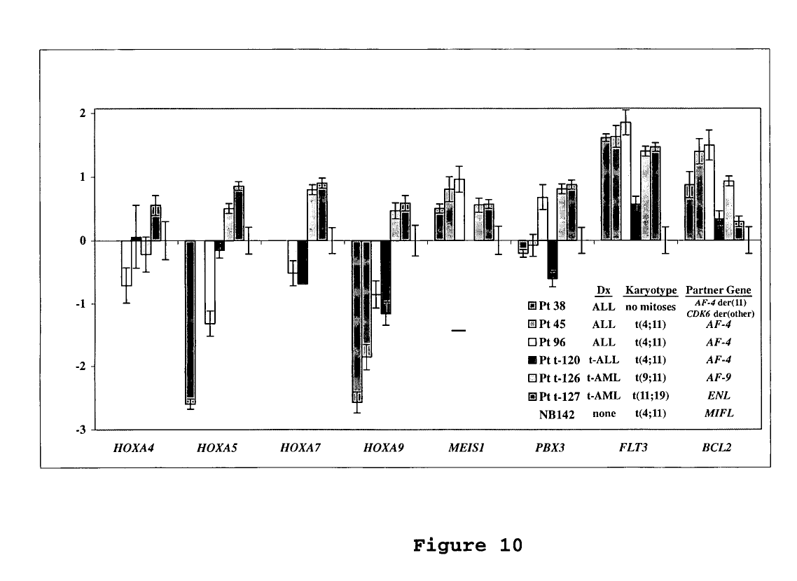

Protein translations across all 6 reading frames of the KIAA0826 transcript were queried with the BLASTP algorithm. This search identified functional homology and amino acid identity with the furry (gene name fry) protein of Drosophila melanogaster (GenBank accession #AAG41424), which is involved in bristle morphogenesis (FIG. 7; Cong et al. (2001) Development 128:2793-2802). Thus the new MLL partner gene appears to encode a heretofore unknown human protein with homology to a Drosophila protein involved in tissue differentiation and cell fate.

MLL Gene Translocation is Detected After Etoposide Treatment

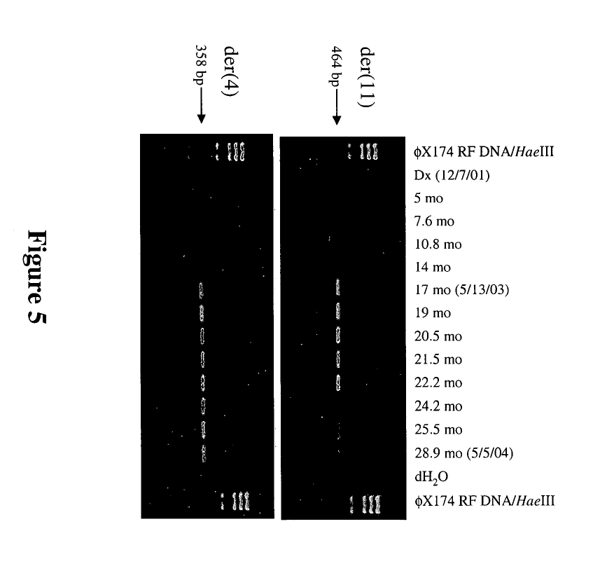

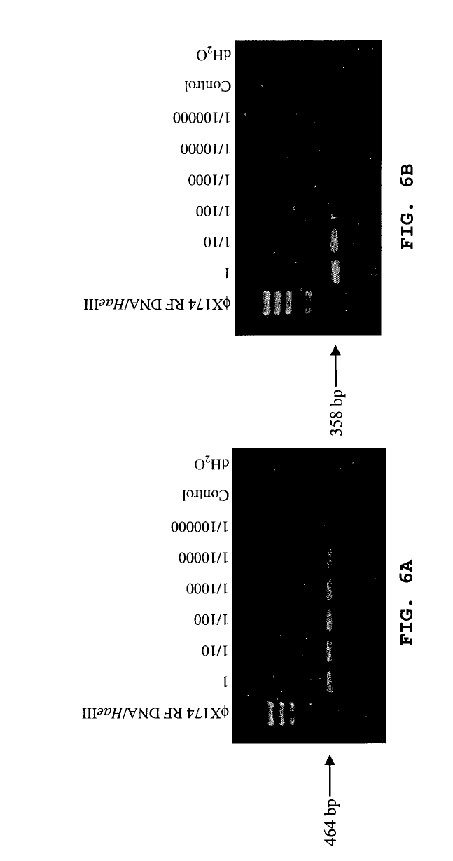

PCR analysis of the der(11) and der(4) breakpoint junctions with clonotypic primers detected translocations at 16 months after neuroblastoma diagnosis (FIG. 5). In addition, each successive bone marrow sample tested contained both the der(11) and der(4) translocation breakpoints as verified by hemi-nested clonotypic PCR and sequencing of the PCR product (FIG. 5). The translocations were not PCR-detectable in the bone marrow samples from diagnosis up to before 16 months. The appearance of the translocations appears to closely correlate with the completion of therapy involving very high cumulative doses of oral etoposide, a known topoisomerase II inhibitor, however other agents that are associated with leukemia were included in the regimen. Bone marrow DNA obtained 21.5 months after neuroblastoma diagnosis was serially diluted 1:10 into peripheral blood DNA from a normal subject. The assay provided a sensitivity of between 1 cell in 10000 and 1 cell in 100000 for the der(11) breakpoint (FIG. 6A). The sensitivity was reduced for the der(4) breakpoint after hemi-nested PCR, between 1 cell in 100 and 1 cell in 1000 (FIG. 6B).

Quantitative Real-Time PCR

The gene expression profile of the bone marrow of patient NB142, which has not shown any evidence of leukemia, was compared against the gene expression profile in the leukemia cells of patients with de novo ALL (p38, p45, p96), treatment-related ALL (t-120), and treatment-related AML (t-126, t-127) (Table 1). HOXA4, HOXA5, HOXA7, HOXA9, MEIS1, PBX3, FLT3, and BCL2 genes were examined and results are relative to patient NB142 using the ΔΔC_{T} comparative method, in triplicate. The four HOX genes studied were expressed at levels less than in the bone marrow of patient NB142 for the patients diagnosed with ALL leukemia (de novo or treatment-related), but the leukemia cells from two t-AML patients had significantly increased levels compared to patient NB142 (except HOXA4 for t-126). p96, a de novo ALL, had extremely high levels of MEIS1 and PBX3 (HOX co-factors) as well as FLT3 and BCL2 compared to patient NB142. Patient NB142 had the lowest detectable levels of MEIS1, FLT3, and BCL2.

TABLE 1
Table 1. Real-time PCR gene expression comparison of NB142 patient with ALL
and AML de novo (p) or treatment-related (t) leukemia patients.

| Cell Line | HOXA4 | HOXA5 | HOXA7 | HOXA9 | MEIS1 | PBX3 | FLT3 | BCL2 |
|---|---|---|---|---|---|---|---|---|
| NB142 | 1.0 | 1.0 | 1.0 | 1.0 | 1.0 | 1.0 | 1.0 | 1.0 |
| p38 | n.d. | <0.1 | n.d. | <0.1 | 3.2 | 0.6 | 40.4 | 7.4 |
| p45 | n.d. | n.d. | n.d. | <0.1 | 6.4 | 0.8 | 42.2 | 24.7 |
| p96 | 0.2 | <0.1 | 0.3 | 0.1 | 9.1 | 4.7 | 70.4 | 31.0 |
| t-120 | 1.2 | 0.7 | 0.2 | 0.1 | n.d. | 0.2 | 3.7 | 2.1 |
| t-126 | 0.6 | 3.2 | 6.3 | 2.9 | 3.6 | 7.5 | 28.9 | 8.4 |
| t-127 | 3.6 | 7.2 | 8.0 | 3.8 | 3.7 | 6.4 | 24.9 | 2.0 |

Results are relative to NB142 (bold) using the ΔΔC_{T} comparative method (18s rRNA used as endogenous control).
n.d.; not detectable.
Results were done in triplicate and dH₂O was run as a negative control.

The gene expression profile of patient NB142 was compared over a period of 6.5 months starting near the end of N8 neuroblastoma chemotherapy (Table 2). HOXA4, HOXA5, HOXA7, HOXA9, MEIS1, PBX3, FLT3, and BCL2 genes were examined and results are relative to the sample date with the lowest detectable expression using the ΔΔC_{T} comparative method, in triplicate. Expression levels of the four HOX genes, MEIS1, and PBX3 decreased significantly following the Jul. 14, 2003 sample date. The levels of FLT3 and BCL2 fluctuated during the sample dates with the lowest levels found on the last sample date, Jan. 28, 2004. In addition, the last sample date, Jan. 28, 2004 had the lowest detectable levels in six out of the eight genes examined.

TABLE 2
Table 2. Real-time PCR gene expression comparison of NB142 patient samples over
a period of 6.5 months mainly after N8 neuroblastoma chemotherapy.

| Sample Date | HOXA4 | HOXA5 | HOXA7 | HOXA9 | MEIS1 | PBX3 | FLT3 | BCL2 |
|---|---|---|---|---|---|---|---|---|
| Jul. 14, 2003 | 1.1 | 22.1 | 12 | 28.9 | 13 | 7.1 | 2.3 | 2.1 |
| Aug. 27, 2003 | n.d. | 1.2 | n.d. | 1.0 | n.d. | 1.0 | 1.0 | 1.5 |
| Sep. 29, 2003 | 1.0 | 1.1 | 1.3 | 1.6 | 1.6 | 1.1 | 1.6 | 2.2 |
| Oct. 15, 2003 | n.d. | 1.7 | 2.4 | 2.9 | 1.2 | 1.6 | 2.5 | 4.3 |
| Dec. 15, 2003 | n.d. | 3.2 | n.d. | 6.5 | n.d. | 5.5 | 1.1 | 1.5 |
| Jan. 28, 2004 | 1.0 | 1.0 | 1.0 | 1.9 | 1.0 | 2.4 | 1.0 | 1.0 |

Results are relative to the sample date with lowest detectable levels (bold) using the ΔΔC_{T} comparative method (18s rRNA used as endogenous control).
n.d.; not detectable.
Results were done in triplicate and dH₂O was run as a negative control.

### Discussion

The patient described hereinabove is of interest for two reasons. First, the behavior of the novel MLL-MIFL translocation is not characteristic of MLL translocations that have been associated with leukemia. Second, the translocation could initially be detected only later during treatment following administration of a high cumulative dose of oral etoposide was administered. Nearly every MLL translocation described to date has been associated with leukemia and, where tested, the resultant der(11) proteins cause leukemia in mice. The patient whom we characterized does not have any clinical evidence of leukemia even though the marrow remains replaced with the MLL-MIFL clone now years from when it first appeared.

The MLL-MIFL fusion has not led to a leukemia phenotype in the patient and so far it appears to have conferred a proliferative advantage without affecting differentiation. Without being bound by theory, there may be several explanations for the differences compared to MLL fusion gene leukemias. The first is that MLL-MIFL differs from other MLL translocations that affect differentiation. This possibility is supported by the different patterns of gene expression in the cells with MLL-MIFL compared to leukemias with MLL translocations. Murine models suggest that transcriptional activation may be a key function of nuclear MLL partner proteins (Ayton et al. (2001) Oncogene, 20:5695-707; Zeisig et al. (2003) Leukemia, 17:359-65; So et al. (2003) Cancer Cell, 3:161-71) and that the role of cytoplasmic MLL partner proteins involves forced MLL dimerization or oligomerization (So et al. (2003) Cancer Cell, 4:99-110), but that altered HOX expression is the final common pathway in MLL fusion protein leukemogenesis regardless of the partner protein (Yokoyama et al. (2004) Mol. Cell. Biol., 24:5639-49).

In a murine retroviral transplantation model of MLL-ENL, constitutive activation of Hoxa9 was essential for leukemogenesis (Ayton et al. (2003) Genes Dev., 17:2298-307). Both HOXA9 and the HOX coregulator gene MEIS1 gene are overexpressed in leukemias with MLL translocations (Armstrong et al. (2002) Nat. Genet., 30:41-7) and these are the key target genes in MLL-ENL mediated cellular immortalization (Zeisig et al. (2004) Mol. Cell. Biol., 24:617-28). Hoxa9 expression influenced the myeloid phenotype but not the incidence of leukemia in a Mll-AF9 transgenic model (Kumar et al. (2004) Blood, 103:1823-8). Leukemic transformation occurred in absence of Hoxa9 and Hoxa7 in a MLL-GAS7 murine model, although the Hox genes influenced the phenotype, latency and penetrance (So et al. (2004) Blood, 103:3192-9).

Alternatively, a murine model has suggested that prenatal and postnatal myeloid cells demonstrate stepwise progression in the pathogenesis of MLL fusion gene leukemia (Johnson et al. (2003) Blood, 101:3229-35). Another second possibility in the patient in the present study implied by the murine model is that there may be a more subtle perturbation of hematopoietic differentiation that either is not yet apparent or at an early stage where it is reversible (Johnson et al. (2003) Blood, 101:3229-35).

Another possibility is that there will be a different latency to emergence of disease. While leukemia has not been diagnosed it remains to be determined if leukemia ultimately will occur because other MLL fusion protein leukemias have shown a long latency as well (e.g., MLL-GMPS). Latency to leukemia in the murine models has suggested that other alterations may be acquired during MLL oncoprotein leukemogenesis in addition to the translocations (Ayton et al. (2001) Oncogene, 20:5695-707; Ayton et al. (2001) Genesis., 30:201-12). The marrow of this patient also exhibits different expression patterns of other genes compared leukemias with MLL translocations with respect to these alterations. For example, although treatment-related cases have not been studied comprehensively, FLT3 ITD mutations and FLT3 overexpression are also characteristic of some subsets of leukemias with MLL translocation (Armstrong et al. (2002) Nat. Genet., 30:41-7; Armstrong et al. (2003) Cancer Cell, 3:173-83; Taketani et al. (2004) Blood, 103:1085-8; Libura et al. (2003) Blood, 102:2198-204). The cells of this patient do not exhibit increased FLT-3 expression. MLL rearranged leukemias also exhibit high BCL-2 expression and are resistant to induced apoptosis. In contrast, the cells with MLL-MIFL have low BCL-2 expression.

In pediatric cases of treatment-related AML following primary neuroblastoma (Megonigal et al. (2000) Proc. Natl. Acad. Sci., 97:2814-9) or primary ALL (Blanco et al. (2001) Proc. Natl. Acad. Sci., 98:10338-43), the MLL translocation was absent in the bone marrow at primary cancer diagnosis but emerged during the treatment, suggesting that treatment caused and did not select for a pre-existing translocation. MLL translocations can be present early during anticancer treatment at low cumulative doses of these agents (Megonigal et al. (2000) Proc. Natl. Acad. Sci., 97:2814-9) or surface later in the treatment. This patient was unlike the prior patients inasmuch as the translocation was not detected until after a very high cumulative dose of oral etoposide was used.

There is heterogeneity in MLL translocation breakpoint distribution in the bcr in treatment-related leukemias in children, but a hotspot region in intron 8 3′ in the bcr has emerged from the molecular cloning of MLL genomic breakpoint junctions (Domer et al. (1995) Leukemia, 9:1305-12; Lovett et al. (2001) Proc. Natl. Acad. Sci., 98:9802-7; Raffini et al. (2002) Proc. Natl. Acad. Sci., 99:4568-73; Langer et al. (2003) Genes Chromosomes Cancer, 36:393-401; Whitmarsh et al. (2003) Oncogene, 22:8448-59; Megonigal et al. (1997) Proc. Natl. Acad. Sci., 94:11583-8; Megonigal et al. (2000) Proc. Natl. Acad. Sci., 97:2814-9; Sobulo et al. (1997) Proc. Natl. Acad. Sci., 94:8732-7; Raffini et al. (2002) Proc. Natl. Acad. Sci., 99:4568-73). The translocation in this patient is not in the hotspot region. In cases in which both genomic breakpoint junctions have been characterized, the sequences reveal precise or near-precise interchromosomal DNA recombinations with gains or losses of no or, more often, a few bases (Domer et al. (1995) Leukemia, 9:1305-12; Megonigal et al. (2000) Proc. Natl. Acad. Sci., 97:2814-9; Lovett et al. (2001) Proc. Natl. Acad. Sci., 98:9802-7; Raffini et al. (2002) Proc. Natl. Acad. Sci., 99:4568-73; Whitmarsh et al. (2003) Oncogene, 22:8448-59; Langer et al. (2003) Genes Chromosomes Cancer, 36:393-401), which was the case in this patient also. The relatively precise interchromosomal DNA recombinations are consistent with the processing of four-base staggered double-stranded breaks from topoisomerase II cleavage (Lovett et al. (2001) Proc. Natl. Acad. Sci., 98:9802-7; Raffini et al. (2002) Proc. Natl. Acad. Sci., 99:4568-73; Whitmarsh et al. (2003) Oncogene, 22:8448-59), and in the present study induced cleavage sites were resolved to form both bp junctions. In contrast, the finding that the dox in the regimen had marginal activity at the translocation bps provides further evidence that the high dose oral etoposide caused the MLL-MIFL translocation. The translocation breakpoints in MLL and MIFL were reciprocally cleaved by topoisomerase II in vitro, and functional, drug-induced DNA topoisomerase II cleavage sites could be resolved to form the breakpoint junctions (Lovett et al. (2001) Proc. Natl. Acad. Sci., 98:9802-7; Whitmarsh et al. (2003) Oncogene, 22:8448-59). The genotoxic catechol and quinone metabolites of etoposide, like etoposide, increased the formation of DNA topoisomerase II cleavage complexes at or near the translocation breakpoints. These data favor a model in which etoposide or etoposide metabolite induced topoisomerase II cleavage was the mechanism of chromosomal breakage leading to the MLL-MIFL translocation.

Recent data from a human primary CD34+ cell model that etoposide induces a full spectrum MLL rearrangements that remain stable after clonal expansion (Libura et al. (2004) Blood, 105:2124-31), give additional evidence for a direct causal relationship between etoposide and translocations. The data from Le Deley coupled with the timing of emergence of the translocation in the patient in this study suggest oral etoposide should be used with caution.

In treatment-related and de novo leukemias with MLL translocations alike, microhomologies of several overlapping bases at/near the translocation breakpoints in MLL and partner genes have suggested DNA damage resolution by the repair mechanism of nonhomologous end-joining (NEJ) (Domer et al. (1995) Leukemia, 9:1305-12; Lovett et al. (2001) Proc. Natl. Acad. Sci., 98:9802-7; Raffini et al. (2002) Proc. Natl. Acad. Sci., 99:4568-73; Whitmarsh et al. (2003) Oncogene, 22:8448-59; Megonigal et al. (2000) Proc. Natl. Acad. Sci., 97:2814-9; Super et al. (1997) Genes Chromosomes Cancer, 20:185-95; Megonigal et al. (1998) Proc. Natl. Acad. Sci., 95:6413-8; Felix et al. (1997) Blood, 90:4679-86; Reichel et al. (1999) Cancer Res., 59:3357-62; Felix et al. (1999) Mol. Diagn., 4:269-83) which also is consistent with the findings at the breakpoint junctions of the MLL-MIFL translocation. Short sequence homologies and loss of 8-13 bases from MLL and 31-36 bases from MIFL suggest imprecise NHEJ repair. The small deletions relative to native MLL and MIFL are consistent with the limited processing that often is required for NHEJ to ensue (Jackson S P (2002) Carcinogenesis, 23:687-96), which has been observed at other translocation breakpoints (Megonigal et al. (2000) Proc. Natl. Acad. Sci., 97:2814-9; Raffini et al. (2002) Proc. Natl. Acad. Sci., 99:4568-73).

Leukemias associated with topoisomerase II poisons have heterogeneous presentations, which are characteristic of the underlying chromosomal translocations. Epipodophyllotoxin-related leukemias with MLL translocations usually are FAB M4 myelomonocytic (FAB M4) or monoblastic (FAB M5) variants of AML (Ratain et al. (1987) Blood, 70:1412-7; Pui et al. (1988) Cancer Res., 48:5348-52), but also can present as other AML subtypes, MDS or ALL (Smith et al. (1994) Med. Pediatr. Oncol., 23:86-98; Felix et al. (1995) Blood, 85:3250-6; Winick et al. (1993) J. Clin. Oncol., 11:209-17).

### EXAMPLE II

### Improved Methods for Detecting Translocation Breakpoints and Characterizing Fusion Genes Resulting Therefrom

The panhandle PCR and panhandle variant PCR for cloning der(11) breakpoint junctions, and the reverse panhandle PCR for cloning the breakpoint junctions of the der(other) chromosomes of MLL translocations have been adapted to handle restriction fragments that were previously too large for PCR-based cloning. Early panhandle work involved rearranged BamHI genomic fragments because the translocation breakpoints are distributed in an 8.3 kb breakpoint cluster region (bcr) defined by the BamHI fragment between exons 5 and 11, and 8.3 kb is a size amenable to PCR-based cloning. In cases where the BamHI genomic fragments were larger than the germline 8.3 kb size, cDNA panhandle PCR was the only way to determine the partner gene of interest. Alternative panhandle and reverse panhandle PCR cloning strategies were developed that take advantage of the BglII restriction sites at MLL intron 6 position 2253 and at intron 10 position 8234.

Previously, the der(11) genomic breakpoint junction fusing MLL intron 7 position 2913 and AF-6 intron 1 position 32501 in the AML of patient 21 was amplified after ligation of the relevant phosphorylated oligonucleotide (p-oligo) to BamHI-digested DNA. Here, original BamHI-based panhandle PCR was employed to validate this breakpoint junction; a 3.7 kb product was obtained and sequencing confirmed the expected breakpoint, also characterized by an “AGGA” insertion. Because the MLL der(11) genornic breakpoint was in intron 7, 3′ of the intron 6 BglII site at position 2253, this genomic breakpoint junction became the test case for the new BglII-based panhandle PCR.

The der(4) genomic breakpoint junction of a t(4;11) translocation in the ALL of patient 45 also was cloned previously by BamHI-based reverse panhandle PCR (Raffini PNAS '02) fusing AF-4 intron 3 position 34864 or 34865 and MLL intron 8 position 6166 or 6167. Based on the known sequence of the genomic breakpoint junction, we determined that a BglII digestion of the genomic DNA from patient 45 would elicit clonable products. Thus, patient 45 became the test case for the new BglII-based reverse panhandle PCR.

The following materials and methods are provided to facilitate the practice of example II.

Characterization of der (11) Genomic Breakpoint Junction using BglII Panhandle Polymerase Chain Reaction

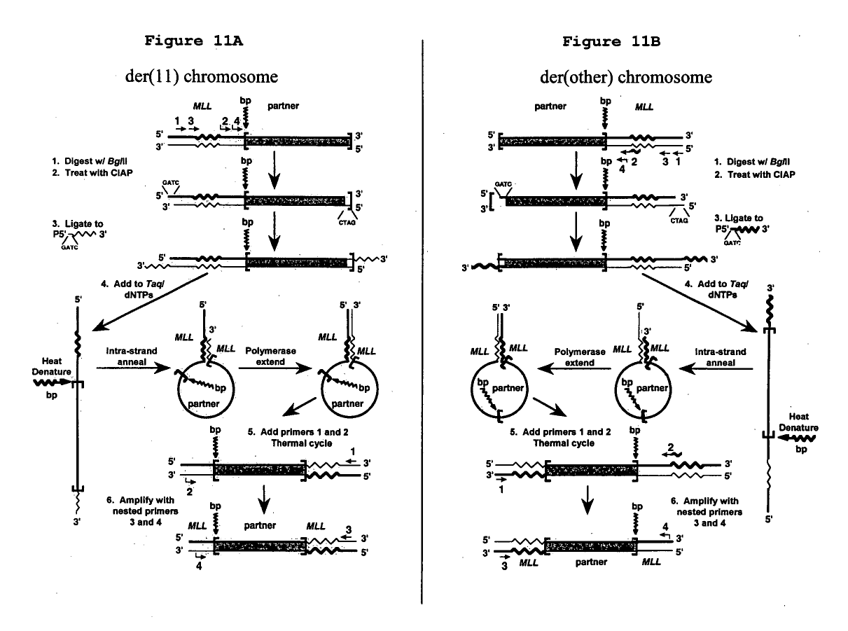

For patient 21, BglII-digested DNA and adapted panhandle PCR was used to amplify the breakpoint from a der(11) chromosome with known 5′ MLL sequence juxtaposed to 3′ unknown partner (FIG. 11A). Step 1: Genomic DNA was digested at 37° C. for 2 hrs with BglII to create a restriction fragment with a 5′ overhang. The 100-μl reaction contained 5 μg of genomic DNA, 20 units of BglII, and 10× React 3 buffer (Invitrogen). 0.05 units of calf intestinal alkaline phosphatase (Boehringer Mannheim Biochemicals) was added to the digested DNA and incubated at 37° C. for 30 minutes. The digested, phosphatase-treated DNA was purified by using a Geneclean III kit (Bio 101) and resuspended in 25 μl dH₂O. Step 2: A single-stranded 5′ phosphorylated oligonucleotide that was antisense to known MLL sequence at positions 2432-2401 in exon 7 (GenBank accession no. U04737) in the bcr was ligated to the 5′ ends of the BglII digested DNA at 4° C. overnight. The sequence was 5′-GAT CGA ACT ATT GCC ATT GGA GAG AGT GCT GAG GAT. The 50-μl ligation reaction contained 16.9 μl of dH₂O, 25 μl of digested, phosphatase-treated DNA (5 μg), 2.1 μl of 5′ phosphorylated oligonucleotide (0.25 μg/μl), 5 μl of 10× ligase buffer, and 1 μl of T4 DNA ligase (1 unit/μl) (Boehringer Mannheim Biochemicals). The DNA was again purified using a Geneclean III kit (Bio 101) and resuspended in 25 μl dH₂O. Step 3: Two microliters of digested, ligated DNA was added to a 43-μl mixture containing 0.5 μl of Taq/Tgo DNA polymerase (5 units/μl), 0.7 μl of 25 mM dNTP mix, and 10 μl 10× PCR buffer (Expand Long Template PCR System, Roche). The mixture was heated to 80° C. for 5 minutes before the DNA was added. The DNA was made single-stranded by heating the reaction mixture at 94° C. for 1 minute. The stem-loop template was formed by a 2 minute ramp to 72° C. and incubated at 72° C. for 30 seconds to promote intrastrand annealing of the ligated oligonucleotide to the complementary sequence in the sense strand and polymerase extension of the recessed 3′ end. Step 4: With the reaction mixture at 80° C., 12.5 pmoles of primer 1 corresponding to MLL exon 7 sense positions 2357-2381 (5′-AAC CAC CTC CGG TCA ATA AGC AGG A-3′) and of primer 2 corresponding to MLL exon 7 sense positions 2442-2467 (5′-AAT TCC AGC AGA TGG AGT CCA CAG GA-3′) was added. Each final 50-μl PCR reaction mixture contained 12.5 pmol each of primers 1 and 2, 350 μM of each dNTP, and 1× PCR buffer. After denaturation at 94° C. for 1 minute, 10 cycles at 94° C. for 10 s and 68° C. for 7 minutes, and 20 cycles at 94° C. for 10 s and 68° C. for 7 minutes (increment, 20 s/cycle) were used, followed by final elongation at 68° C. for 7 minutes. Step 5: Nested PCR was performed using 1 μl of the product of the preceding PCR as a template. The PCR product was added to a 49-μl mixture containing 0.5 μl of Taq/Tgo DNA polymerase (5 units/ul), 0.7 μl of 25 mM dNTP mix, 10 μl 10× PCR buffer (Expand Long Template PCR System, Roche), 12.5 pmoles of primer 3 corresponding to MLL exon 7 sense positions 2377-2400 (5′-CAG GAG AAT GCA GGC ACT TTG AAC-3′), and of primer 4 corresponding to MLL exon 7 sense positions 2460-2484 (5′-CCA CAG GAT CAG AGT GGA CTT TAA G-3′). The mixture was heated to 80° C. for 5 minutes before the PCR product was added. Each final 50-μl PCR reaction mixture contained 12.5 pmol each of primers 3 and 4, 350 μM of each dNTP, and 1× PCR buffer. Nested PCR conditions were the same as in the initial PCR.

The BglII reverse panhandle PCR products were subcloned into the pCR 2.1-TOPO vector using a TOPO TA Cloning Kit (Invitrogen). The nested PCR product was purified using a Geneclean III kit and resuspended in 10 μl dH₂O (Bio 101). Purified PCR product (2.5 μl) was added to a mixture containing 1 μl salt solution (1.2 M NaCl, 0.06 M MgCl₂), 1.5 μl dH₂O, and 1 μl TOPO vector (pCR 2.1-TOPO) and incubated at room temperature for 15 minutes. Two microliters of the TOPO cloning reaction was added to a vial of One Shot DH5α-T1 Chemically Competent Cells (Invitrogen). The reaction was incubated on ice for 30 minutes, followed by heat-shocking the cells for 30 seconds at 42° C. The heat-shocked cells were put on ice for 2 minutes prior to adding 300 μl S.O.C. medium (Invitrogen) and allowed to recover at 37° C. for 1 hour with shaking (225 rpm). 50 or 300 μl of the transformation reaction was plated on LB/agar plates containing carbenicillin (100 μg/ml) and incubated overnight at 37° C. Individual transformants were grown up for 5 hours at 37° C. in 2 ml of LB broth containing carbenicillin (100 μg/ml).

We used PCR to identify recombinant plasmids containing products of BglII panhandle PCR. Two microliters of the saturated 2 ml cultures were amplified in PCRs containing 0.5 μl of Taq/Tgo DNA polymerase (5 units/μl), 0.7 μl of 25 mM dNTP mix, 10 μl 10× PCR buffer (Expand Long Template PCR System Roche), 12.5 pmoles of primer 3 corresponding to MLL exon 7 sense positions 2377-2400 (5′-CAG GAG AAT GCA GGC ACT TTG AAC-3′), and of primer 4 corresponding to MLL exon 7 sense positions 2460-2484 (5′-CCA CAG GAT CAG AGT GGA CTT TAA G-3′). Each final 50-μl PCR screen reaction mixture contained 12.5 pmol each of primers 3 and 4, 350 μM of each dNTP, and 1× PCR buffer. PCR screen conditions were the same as in the initial PCR. Agarose gel electrophoresis of the products identified transformants containing the recombinant plasmid DNA of interest, which then were grown in 4-ml cultures for plasmid preparation and automated sequencing.

Characterization of der (other) Genomic Breakpoint Junction using BglII Reverse Panhandle Polymerase Chain Reaction

For patient 45, we used BglII-digested DNA and adapted reverse panhandle PCR to amplify the breakpoint from a der(other) chromosome with known 3′ MLL sequence juxtaposed to 5′ unknown partner (FIG. 11B). Step 1: Genomic DNA was digested at 37° C. for 2 hrs with BglII to create a restriction fragment with a 5′ overhang. The 100-μl reaction contained 5 μg of genomic DNA, 20 units of BglII, and 10× React 3 buffer (Invitrogen). 0.05 units of calf intestinal alkaline phosphatase (Boehringer Mannheim Biochemicals) was added to the digested DNA and incubated at 37° C. for 30 minutes. The digested, phosphatase-treated DNA was purified by using a Geneclean III kit (Bio 101) and resuspended in 25 μl dH₂O. Step 2: A single-stranded 5′ phosphorylated oligonucleotide that was homologous to known MLL sequence at positions 8004-8035 in exon 10 (GenBank accession no. U04737) in the bcr was ligated to the 3′ ends of the BglII digested DNA at 4° C. overnight. The sequence was 5′-GAT CAC CCT GAG TGC CTG GGA CCA AAC TAC CCC ACC. The 50-μl ligation reaction contained 16.9 μl of dH₂O, 25 μl of digested, phosphatase-treated DNA (5 μg), 2.1 μl of 5′ phosphorylated oligonucleotide (0.25 μg/μl), 5 μl of 10× ligase buffer, and 1 μl of T4 DNA ligase (1 unit/μl) (Boehringer Mannheim Biochemicals). The DNA was again purified using a Geneclean III kit (Bio 101) and resuspended in 25 μl dH₂O. Step 3: Two microliters of digested, ligated DNA was added to a 43-μl mixture containing 1 μl of Elongase Enzyme Mix (1 unit/μl), 1 μl of 10 mM dNTP mix, 5 μl of 5× Buffer A, and 5 μl of 5× Buffer B (Elongase Amplification System, Invitrogen). The mixture was heated to 80° C. for 5 minutes before the DNA was added. The DNA was made single-stranded by heating the reaction mixture at 94° C. for 1 minute. The stem-loop template was formed by a 2 minute ramp to 72° C. and incubated at 72° C. for 30 seconds to promote intrastrand annealing of the ligated oligonucleotide to the complementary sequence in the antisense strand and polymerase extension of the recessed 3′ end (Felix et al. (1997) Blood, 90:4679-86). Step 4: With the reaction mixture at 80° C., 12.5 pmoles of primer 1 corresponding to MLL intron 10 antisense positions 8089-8063 (5′-TAA AAG AGC ATC ATG TGT ATA ACT CAC-3′) and of primer 2 corresponding to MLL exon 10 antisense positions 8017-7991 (5′-CAG GCA CTC AGG GTG ATA GCT GTT TCG-3′) was added. Each final 50-μl PCR reaction mixture contained 12.5 pmol each of primers 1 and 2, 200 μM of each dNTP, and 1× PCR buffer. After denaturation at 94° C. for 1 minute, 10 cycles at 94° C. for 10 s and 68° C. for 7 minutes, and 20 cycles at 94° C. for 10 s and 68° C. for 7 minutes (increment, 20 seconds/cycle) were used, followed by final elongation at 68° C. for 7 minutes. Step 5: Nested PCR was performed using 1 μl of the product of the preceding PCR as a template. The PCR product was added to a 49-μl mixture containing 1 μl of Elongase Enzyme Mix (1 unit/μl), 1 μl of 10 mM dNTP mix, 5 μl of 5× Buffer A, 5 μl of 5× Buffer B (Elongase Amplification System, Invitrogen), 12.5 pmoles of primer 3 corresponding to MLL exon 10 antisense positions 8062-8036 (5′- CCA GAC TTT CTT CTT CTT TGT GGG TTT-3′), and of primer 4 corresponding to MLL exon 10 antisense positions 8000-7971 (5′-AGC TGT TTC GGC ACT TAT TAC ACT CCA GCA-3′). The mixture was heated to 80° C. for 5 minutes before the PCR product was added. Each final 50-μl PCR reaction mixture contained 12.5 pmol each of primers 3 and 4, 200 μM of each dNTP, and 1× PCR buffer. Nested PCR conditions were the same as in the initial PCR.

The BglII reverse panhandle PCR products were subcloned into the pCR 2.1-TOPO vector using a TOPO TA Cloning Kit (Invitrogen). The nested PCR product was purified using a Geneclean III kit and resuspended in 10 μl dH₂O (Bio 101). Purified PCR product (2.5 μl) was added to a mixture containing 1 μl salt solution (1.2 M NaCl, 0.06 M MgCl₂), 1.5 μl dH₂O, and 1 μl TOPO vector (pCR 2.1-TOPO) and incubated at room temperature for 15 minutes. Two microliters of the TOPO cloning reaction was added to a vial of One Shot DH5α-T1 Chemically Competent Cells (Invitrogen). The reaction was incubated on ice for 30 minutes, followed by heat-shocking the cells for 30 seconds at 42° C. The heat-shocked cells were put on ice for 2 minutes prior to adding 300 μl S.O.C. medium (Invitrogen) and allowed to recover at 37° C. for 1 hour with shaking (225 rpm). 50 or 300 μl of the transformation reaction was plated on LB/agar plates containing carbenicillin (100 μg/ml) and incubated overnight at 37° C. Individual transformants were grown up for 5 hours at 37° C. in 2 ml of LB broth containing carbenicillin (100 μg/ml).

We used PCR to identify recombinant plasmids containing products of BglII reverse panhandle PCR. Two microliters of the saturated 2 ml cultures were amplified in PCRs containing 1 μl of Elongase Enzyme Mix (1 unit/μl), 1 μl of 10 mM dNTP mix, 5 μl of 5× Buffer A, 5 μl of 5× Buffer B (Elongase Amplification System, Invitrogen), 12.5 pmoles of primer 3 corresponding to MLL exon 10 antisense positions 8062-8036 (5′- CCA GAC TTT CTT CTT CTT TGT GGG TTT-3′), and of primer 4 corresponding to MLL exon 10 antisense positions 8000-7971 (5′-AGC TGT TTC GGC ACT TAT TAC ACT CCA GCA-3′). Each final 50-μl PCR screen reaction mixture contained 12.5 pmol each of primers 3 and 4, 200 μM of each dNTP, and 1× PCR buffer. PCR screen conditions were the same as in the initial PCR. Agarose gel electrophoresis of the products identified transformants containing the recombinant plasmid DNA of interest, which then were grown in 4-ml cultures for plasmid preparation and automated sequencing.

### Results

Characterization of der(11) Genomic Breakpoint Junction in Patient 21

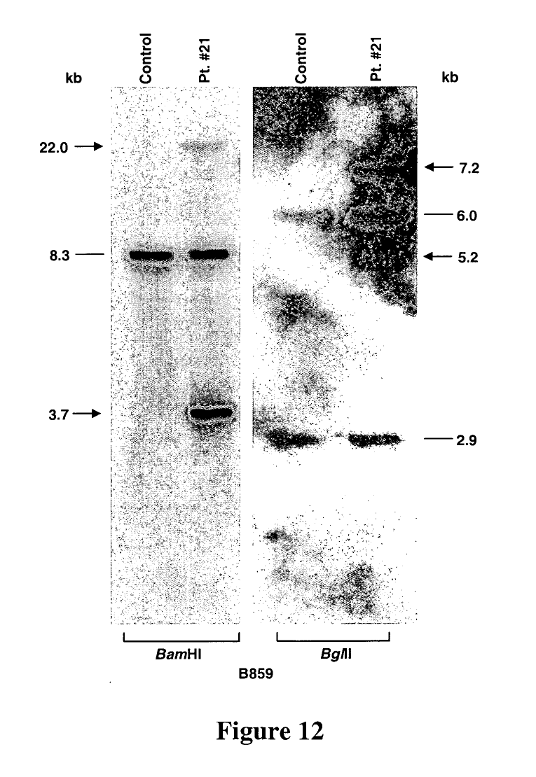

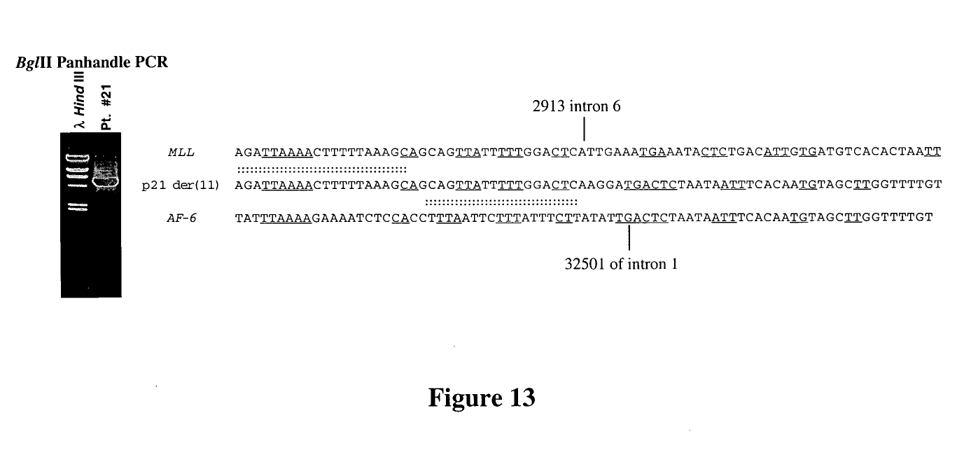

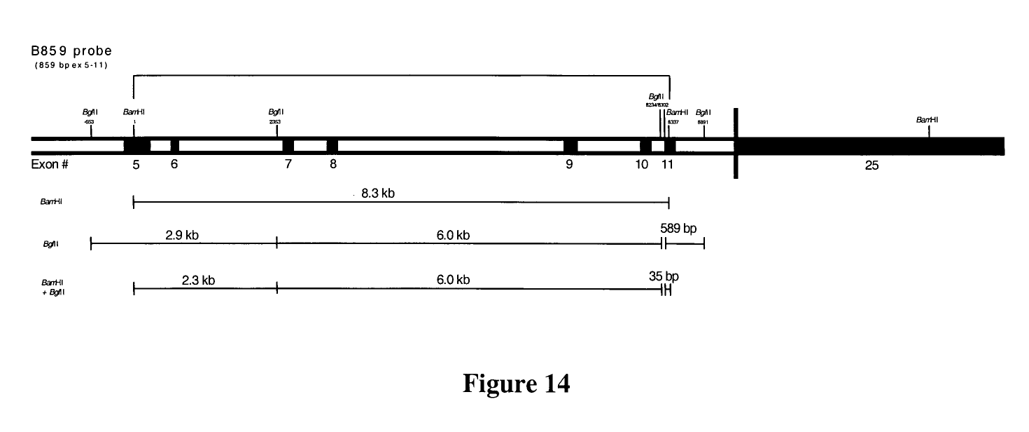

Southern blot analysis of BamHI-digested DNA revealed 22.0 kb and 3.7 kb MLL bcr rearrangements (left) and BglII-digested DNA revealed 7.2 kb and 5.2 kb MLL bcr rearrangements (right) in the infant AML with t(6;11)(q27;q23) of patient 21 (FIG. 12). BamHI panhandle PCR was employed to verify the genomic breakpoint junction and partner gene which had previously been determined (data not shown). BglII panhandle PCR was used and identified the der(11) genomic breakpoint junction and partner gene, AF-6 (FIG. 13, right). The 5114 bp panhandle PCR product shown in FIG. 13 (left), suggested that the 5.2 kb and 7.2 kb rearrangements on the Southern blot (FIG. 12) were from the der(11) and der(6) chromosomes, respectively. Sequencing of TOPO TA cloning generated subclones revealed the MLL der(11) breakpoint at position 2913 in intron 7 (GenBank accession no. U04737), which is 5′ in the bcr (FIG. 14). The der(11) breakpoint in the partner gene corresponded to position 32501 in AF-6 intron 1 (GenBank accession no. U02478). PCR subclones generated from BamHI-digested DNA revealed the MLL der(11) breakpoint to also be at position 2913 in intron 7, where the BglII-digested DNA suggested (FIG. 13, right), thus verifying the BglII panhandle PCR technique as functioning properly.

Characterization of der(4) Genomic Breakpoint Junction in Patient 45

Previous work showed a Southern blot analysis of BamHI-digested DNA which revealed 6.8 kb and 2.1 kb MLL bcr rearrangements in the infant ALL with t(4;11)(q21;23) of patient 45 (Raffini PNAS '02). BamHI panhandle and reverse panhandle PCR techniques found the MLL der(11) and der(4) breakpoints, respectively. The MLL der(4) genomic breakpoint was at position 6166 or 6167 in intron 8 and the der(4) genomic breakpoint was at position 34864 or 34865 in AF-4 intron 3. “A” residues in both genes at the genomic breakpoint junction did not allow for a precise translocation (Raffini). Based on the work by Raffini et al., it was determined that digesting this patient's DNA with BglII would enable cloning of the der(4) using BglII reverse panhandle PCR.

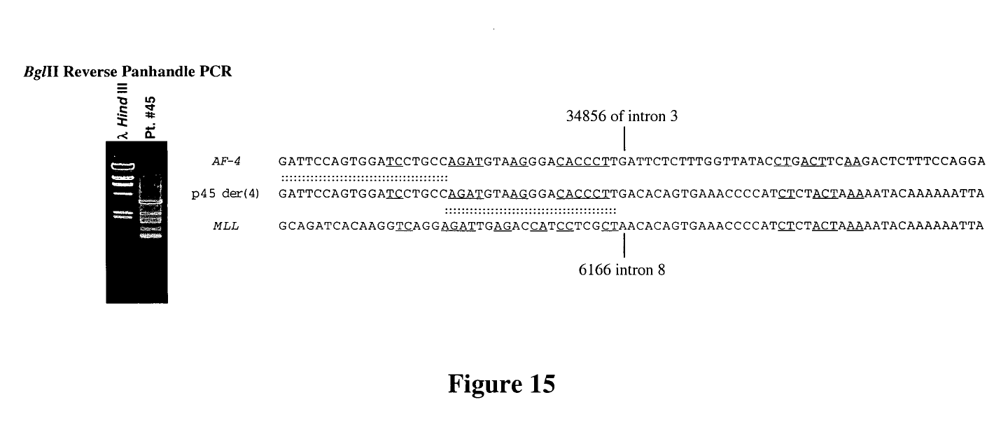

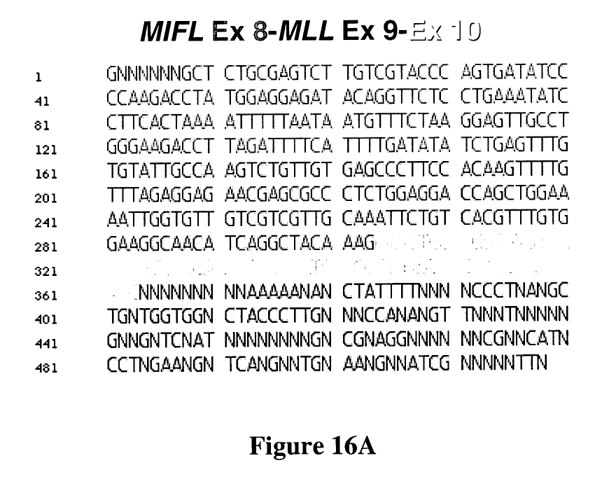

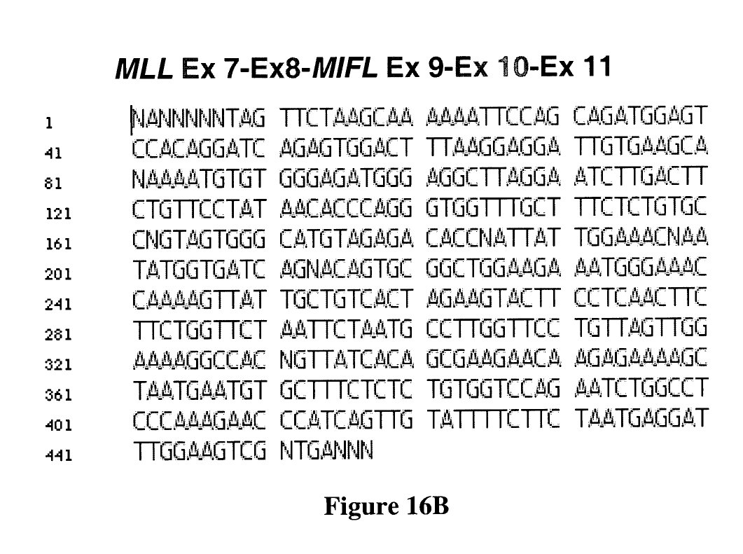

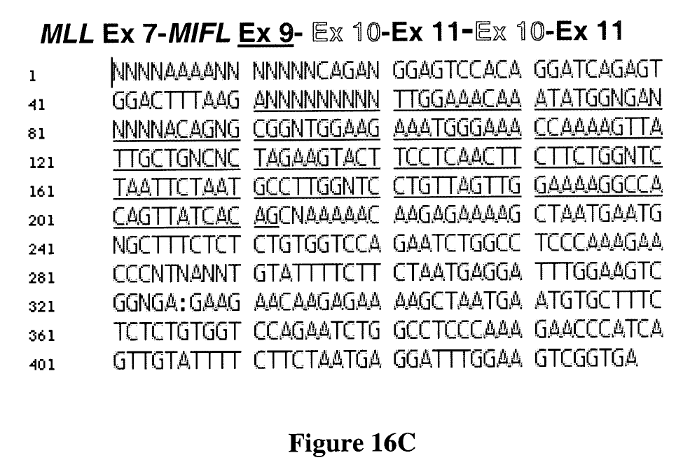

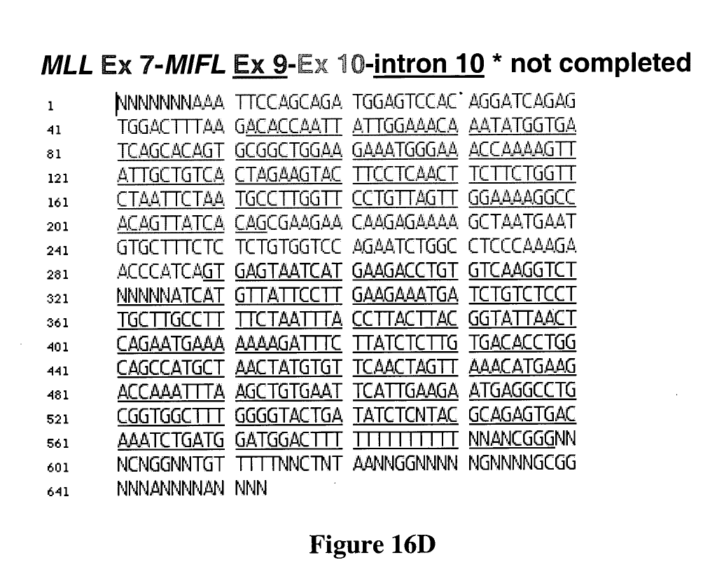

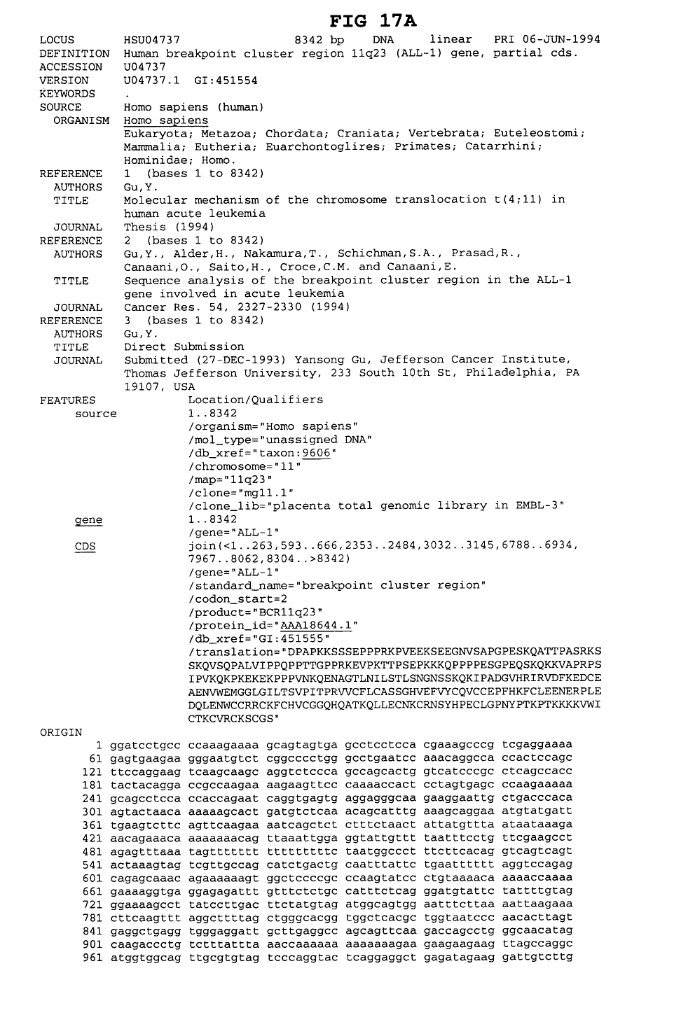

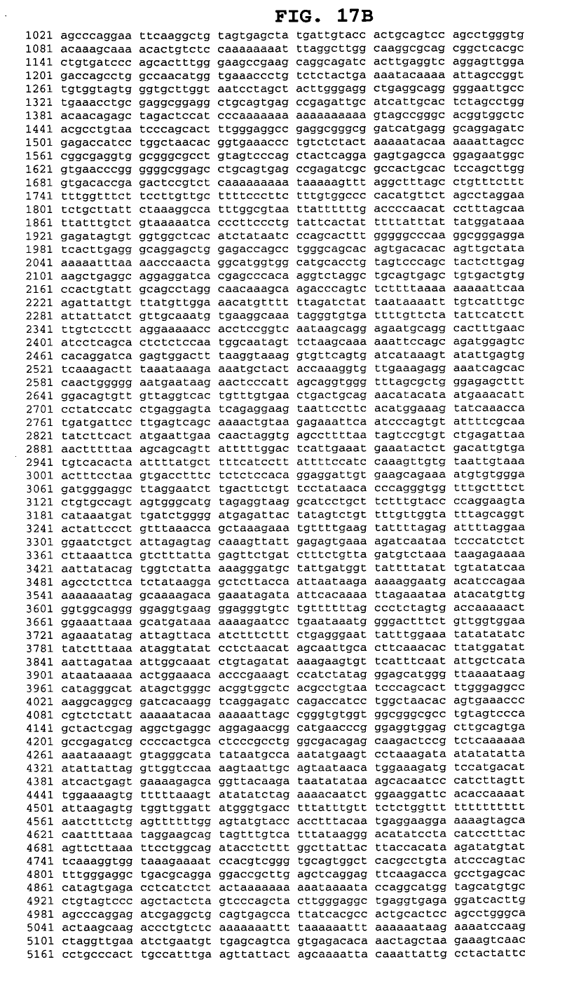

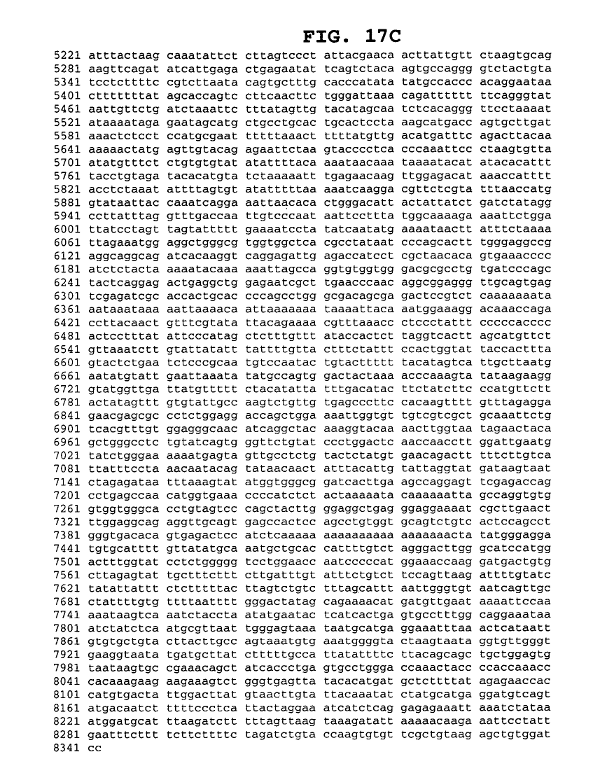

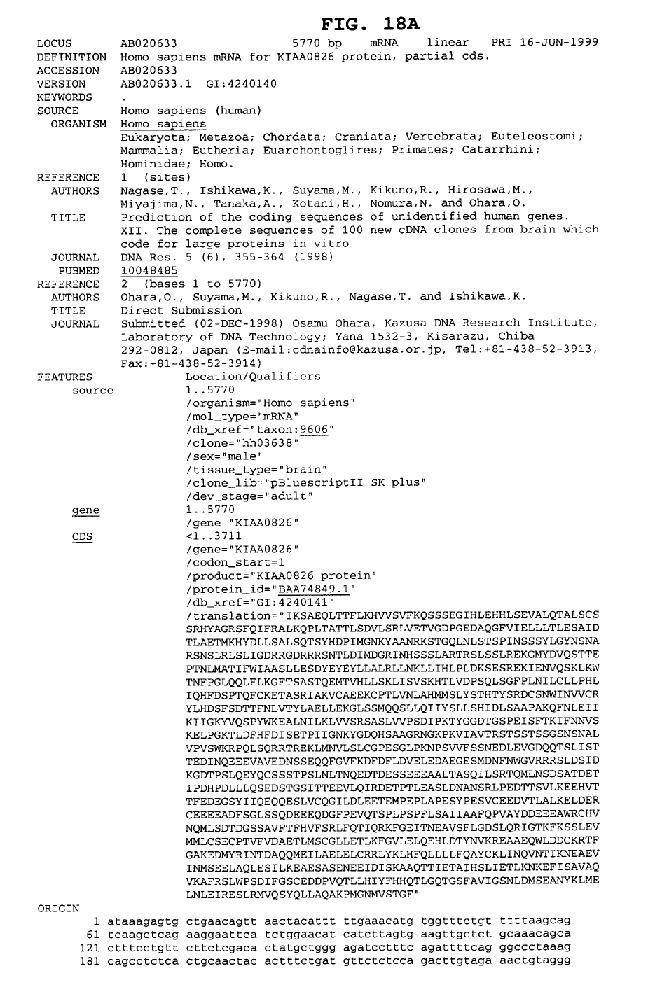

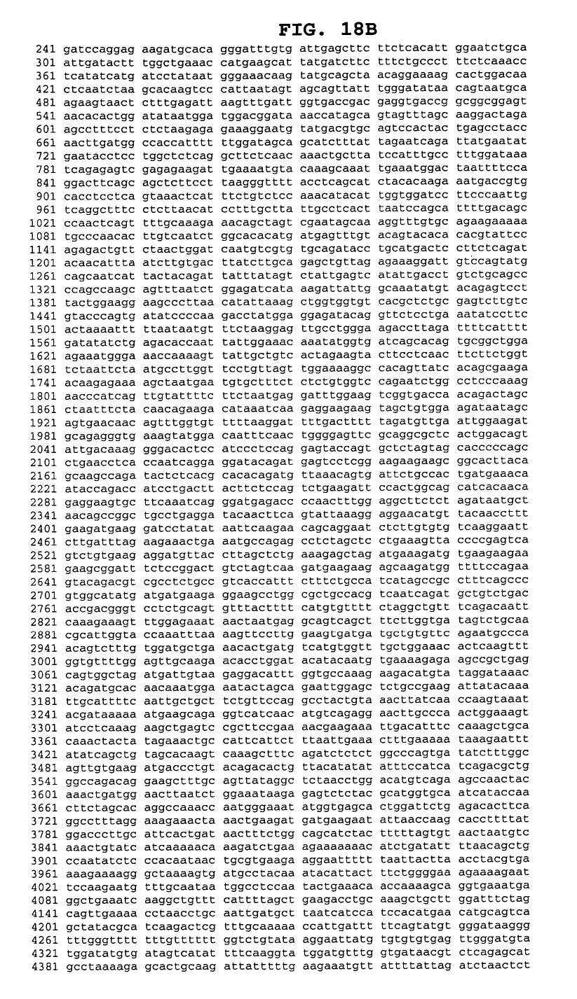

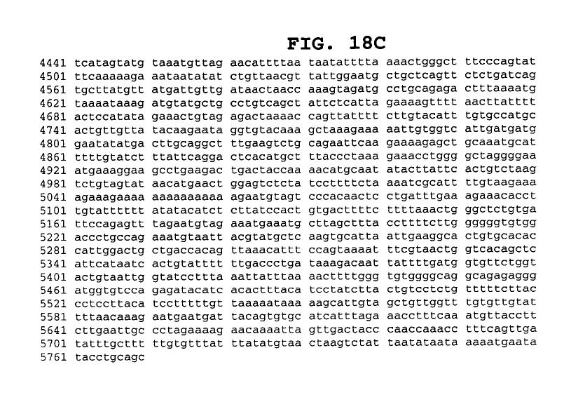

BglII reverse panhandle PCR identified the der(4) genomic breakpoint junction (FIG. 15, right). The 3470 bp reverse panhandle PCR product shown in FIG. 15 (left), suggested that the 4.8 kb and 3.7 kb rearrangements hypothesized to appear on Southern blot would be from the der(11) and der(4) chromosomes, respectively. Sequencing of TOPO TA cloning generated subclones revealed the MLL der(4) breakpoint at position 6166 or 6167 in intron 8 (GenBank accession no. U04737), which is 3′ in the bcr (FIG. 14). The der(4) breakpoint in the partner gene corresponded to position 34855 or 34856 in AF-4 intron 3 (GenBank accession no. L13773). Our results match those of Raffini et al. using BamHI-digested DNA with reverse panhandle PCR, thus verifying the BglII reverse panhandle PCR technique as functioning properly.

### Discussion

BamHI-digested DNA has been used for more than 8 years to clone genomic breakpoint junctions involving MLL and one of its many partner genes using panhandle based PCR techniques. The four major techniques which have been developed: panhandle, variant panhandle, cDNA panhandle, and reverse panhandle PCR, have all been met with great success (Felix et al. (1997) Blood, 90:4679-86; Megonigal et al. (1997) Proc. Natl. Acad. Sci., 94:11583-8; Megonigal et al. (1998) Proc. Natl. Acad. Sci., 95:6413-8; Megonigal et al. (2000) Proc. Natl. Acad. Sci., 97:9597-602; Raffini et al. (2002) Proc. Natl. Acad. Sci., 99:4568-73; Megonigal et al. (2000) Proc. Natl. Acad. Sci., 97:2814-9; Whitmarsh et al. (2003) Oncogene, 22:8448-59; Slater et al. (2002) Oncogene, 21:4706-14; Lovett et al. (2001) Proc. Natl. Acad. Sci., 98:9802-7; Pegram et al. (2000) Blood, 96:4360-2). Utilizing the MLL bcr with BamHI-digested DNA takes advantage of the 8.3 kb region between exons 5 and 11, a perfect size for PCR cloning. Target sequences have been amplified up to 8.3 kb in size (Felix et al. (1997) Blood, 90:4679-86; Megonigal et al. (1997) Proc. Natl. Acad. Sci., 94:11583-8), but a limitation occurs once the rearrangement becomes too large for current PCR-based cloning. Using BglII-digested DNA enables PCR-based cloning in cases where the rearrangements are too large after BamHI digestion.

Using BglII-digested DNA, we have successfully cloned the der(11) chromosome genomic breakpoint junction and identified the partner gene, AF-6, of patient 21 using BglII panhandle PCR. The genomic breakpoint is downstream (3′) of the BglII restriction site in intron 6 of MLL, which allows for the cloning of potential novel partner genes otherwise unclonable using BamHI-based panhandle assays. The primers used for the BglII panhandle PCR technique are located in exon 7 and will only work if the breakpoint junction is 3′ of this region. If the breakpoint junction lies in exon 7 or intron 6, which is a possibility, then BglII panhandle PCR may not function well and another method could be utilized. Using BglII-digested DNA removes 2.3 kb of the MLL bcr from the rearrangement, thus potentially shortening the product being cloned. BglII is a very common restriction site, more so than BamHI at least in AF-4 and AF-6, so the chances that BglII will shorten the partner gene are increased.

BglII reverse panhandle PCR worked to amplify the der(other) chromosome joining the 5′ end of the unknown partner gene to 3′ end of the MLL bcr. Patient 45 was used as a test case for this method and the partner gene, AF-4, and genomic breakpoint junction was successfully cloned using BglII-digested DNA with precise results when compared to previous work using BamHI-digested DNA. The primers used for the BglII reverse panhandle PCR technique are located in exon/intron 10 and will only work if the breakpoint junction is 5′ of this region. Breakpoint junctions in intron 10 have not been reported and this technique will work for any der(other) chromosome translocation as long the clonable size remains reasonable.

The cloning of MLL genomic breakpoint junctions has been improved with the introduction of these novel panhandle techniques. For those patients where the rearranged restriction fragments of MLL and partner gene sequences pose a problem due to size, BglII-digested DNA can be used to try and improve the results for both the der(11) and der(other) chromosome translocations. The addition of these novel techniques involving BglII-digested DNA only enhances the scope of knowledge that can be learned from these MLL translocations. The original panhandle PCR using a BamHI digested template is a definitive approach to better understand the types of MLL breakpoint junctions and to amplify translocations with unknown partner genes. Using BglII-digested DNA now provides an alternative DNA template that enables a new panhandle PCR approach to better understand how MLL translocations lead to leukemia or how, when identified as early disease biomarkers, may be used for disease prevention.

While certain of the preferred embodiments of the present invention have been described and specifically exemplified above, it is not intended that the invention be limited to such embodiments. Various modifications may be made thereto without departing from the scope and spirit of the present invention.

What is claimed is:

1. A nucleic acid molecule comprising a translocation joining MLL (U04737) and MIFL (AB020633).

2. The reciprocal nucleic acid molecule of the translocation of claim 1, on the der(4) chromosome

3. The der(11) and der(4) fusion transcripts from the nucleic acid in claim 1.

4. The fusion transcripts of claim 3, wherein said transcripts are selected from the group consisting of SEQ ID NOs: 1-4.

5. The protein product(s) from the expression of the nucleic acid of claim 1.

6. The protein products of the fusion transcripts of claim 4.

7. A method for screening compounds for the ability to modulate proliferation and/or transformation of a cell comprising:
a) providing cells comprising the nucleic acid molecule of claim 1;
b) incubating said cells with said compound; and
c) monitoring proliferation of said cells;
wherein a change in the proliferation/transformation of said cells incubated with said compound as compared to cells not treated with said compound is indicative of the ability of said compound to modulate proliferation of said cells.

8. The method of claim 7, wherein said compound decreases the proliferation of said cells.

9. The method of claim 7, wherein said compound increases the proliferation of said cells.

10. A method of immortalizing cells in culture, said method comprising introduction of the nucleic acid molecule of claim 1 into to said cells.

11. A transgenic animal comprising the nucleic acid molecule of claim 1.

12. The transgenic animal of claim 11 which is a mouse.

13. A method for screening compounds for the ability to modulate leukemogenesis comprising:
a) providing a transgenic animal of claim 11;
b) administering said compound to said animal; and
c) monitoring said animal for leukemogenesis.

13. An immortalized cell line, said cells comprising the nucleic acid molecule of claim 1.

14. Isolated primary host cells comprising the nucleic acid of claim 1.

15. The cells of claim 14, obtained from a patient.

16. The cells of claim 14, which have been subjected to mutagenesis.

17. The cells of claim 16 which have been mutagenized by a method selected from the group consisting of insertional mutagenesis, exposure to ionizing radiation, exposure to ethyl nitrosourea (ENU) and exposure to ethyl methane sulphonate (EMS).

18. The cell line of claim 13, wherein cells are obtained from a patient and are selected from the group consisting of bone marrow cells and blood cells.

19. A method for the screening cells for genetic changes associated with the onset of leukemia, comprising:
a) passaging the cells of claim 15 in a NOD-SCID mouse;
b) assessing said mice for the development of leukemia;
c) isolating said cells from mice which develop leukemia;
d) performing proteomic or genomic analysis comparing the cells of step c) with cells isolated from said patient to identify proteomic or genetic changes associated with the onset of leukemia.

20. The method of claim 19, wherein said cells have been subjected to mutagenesis prior to passaging in said mice.

21. A method for screening for MLL-MIFL fusion nucleic acid sequences in human subjects, comprising:
a) isolating nucleic acid from cells obtained from said patient;
b) contacting said nucleic acid with a probe which hybridizes to a sequence as claimed in claim 1 under conditions suitable for hybridization to occur;
c) detecting hybridization if present, wherein hybridization indicates the presence of a MLL-MIFL fusion transcript in said patient.

22. The method of claim 21, wherein said probe hybridizes to a sequence selected from the group consisting of SEQ ID NO: 1, SEQ ID NO: 2, SEQ ID NO: 3 and SEQ ID NO: 4.

23. The method of claim 21, wherein said cells are blood or bone marrow cells.

24. The method of claim 21, wherein said isolated nucleic acid is analyzed via BgII panhandle PCR.
